(19) United States
(12) Patent Application Publication
(10) Pub. No.: US 2025/0179416 A1
(43) Pub. Date: Jun. 5, 2025

(54) CHARACTERIZING SPERM AND CORRELATING THEIR DNA

(71) Applicant: Anya L. Getman, Sandy, OR (US)

(72) Inventor: Anya L. Getman, Sandy, OR (US)

(21) Appl. No.: 19/037,579

(22) Filed: Jan. 27, 2025

Related U.S. Application Data
(63) Continuation-in-part of application No. 16/817,610, filed on Mar. 13, 2020.
(60) Provisional application No. 62/827,199, filed on Apr. 1, 2019.

(51) Int. Cl. C12M 1/00; C12M 1/26

(52) U.S. Cl. CPC C12M 47/04; C12M 27/20; C12M 29/06; C12M 33/04

(57) ABSTRACT

A sorting track for sorting living sperm includes a housing that defines at least two cubicles, an initial placement location, and a channel fluidly coupled to the at least two cubicles and the initial placement location such that the at least two cubicles each feature a unique set of cubicle characteristics, an extraction portal, and an entrance fluidly coupled to the channel. Cubicle characteristics may include one or more architectural characteristics selected from the group consisting of: a distance from the initial placement location, a cubicle shape, a cubicle volume, a cubicle position within the housing relative to the channel, an entrance size, and an entrance shape. Cubicle characteristics may also include stimulus characteristics selected from the group consisting of: a wavelength of light, a light intensity, a radioactive radiation intensity, an electromagnetic field, a chemical treatment, a pressure, a sound, and a temperature.

### CROSS REFERENCE TO RELATED APPLICATION

This application is a continuation-in-part of U.S. Nonprovisional application Ser. No. 16/817,610, filed Mar. 13, 2020, which claims priority to U.S. Provisional Application Ser. No. 62/827,199, which was filed on Apr. 1, 2019, the contents of each of which are herein incorporated by reference in their entirety for all purposes.

### TECHNICAL FIELD

The field of this disclosure relates generally to classifying and sorting sperm by a variety of characteristics and, in particular, to profiling sperm DNA based on those characteristics.

### BACKGROUND INFORMATION

Every spermatozoa (sperm) is different based on a natural recombination process during sperm production that includes both recombination and mutation. Some prospective parents would like to choose the gender of their offspring and techniques have been developed to separate sperm by the genetic gender of the chromosomes that they carry. Moreover, sperm can be readily separated based on genetic gender without adversely affecting the sperm. In particular, this separation process is accomplished with centrifugation that causes no significant genetic damage. Genetic sex-based selection of sperm for egg (ovum) fertilization has been employed for creating viable embryos for a long time and is no longer as controversial. Sperm DNA and semen components have also been evaluated with respect to enhancing the fertilization process.

### OVERVIEW OF DISCLOSURE

Some prospective parents would like to choose certain other genetic traits of their offspring. However, gene editing of fertilized human eggs (ova) is currently controversial, and DNA analysis of a spermatozoon currently entails killing the spermatozoon to analyze the DNA. The killed spermatozoon cannot be used for ovum fertilization.

In some aspects, the techniques described herein relate to a sorting track for sorting living sperm, including: a housing defining at least two cubicles, an initial placement location, and a channel fluidly coupled to the at least two cubicles and the initial placement location; wherein the at least two cubicles each include a unique set of cubicle characteristics, an extraction portal, and an entrance fluidly coupled to the channel; and wherein the unique set of cubicle characteristics of the at least two cubicles each include one or more architectural characteristics selected from the group consisting of: a distance from the initial placement location, a cubicle shape, a cubicle volume, a cubicle position within the housing relative to the channel, an entrance size, and an entrance shape.

In some aspects, the techniques described herein relate to a sorting track, wherein the unique set of cubicle characteristics of the at least two cubicles each additionally include one or more stimulus characteristics selected from the group consisting of: a wavelength of light, a light intensity, a radioactive radiation intensity, an electromagnetic field, a chemical treatment, a pressure, a sound, and a temperature.

In some aspects, the techniques described herein relate to a sorting track, wherein the one or more stimulus characteristics are present in the at least two cubicles or at or near the entrance of the at least two cubicles.

In some aspects, the techniques described herein relate to a sorting track, further including a sorting track controller configured to selectively vary the one or more stimulus characteristics of the at least two cubicles.

In some aspects, the techniques described herein relate to a sorting track, wherein the housing defines two to one hundred cubicles.

In some aspects, the techniques described herein relate to a sorting track, wherein the cubicle shape may be at least partially conical shape or maze-shaped.

In some aspects, the techniques described herein relate to a sorting track, wherein the cubicle shape may include inner obstacles.

In some aspects, the techniques described herein relate to a sorting track, wherein at least one of the at least two cubicles further includes a camera.

In some aspects, the techniques described herein relate to a sorting track, wherein the housing includes at least one piezoelectrically activated wall for varying a pressure with the channel or within the cubicle as a stimulus characteristic.

In some aspects, the techniques described herein relate to a sorting track, the one or more stimulus characteristics is a chemical treatment, and wherein the chemical treatment includes a sperm attractant or a sperm repellant.

In some aspects, the techniques described herein relate to a sorting track, wherein the chemical treatment includes Lily of the Valley cardiac glycosides, convallotoxin, convallatoxol, convalloside, convallatoxoloside, convallarin, glucoconvalloside, or convallamarin.

In some aspects, the techniques described herein relate to a sorting track, wherein the entrance of at least one of the at least two cubicles further includes an entrance baffle.

In some aspects, the techniques described herein relate to a sorting track, wherein the entrance of at least one of the at least two cubicles further includes a valve.

In some aspects, the techniques described herein relate to a sorting track, wherein the housing further defines an access portal located on a second end of the channel opposite a first end, wherein the first end of the channel is fluidly coupled to the initial placement location.

In some aspects, the techniques described herein relate to a sorting track, wherein the channel has a generally spiral shape.

In some aspects, the techniques described herein relate to a sorting track, wherein the channel includes a first channel and a second channel; wherein the first channel spirals in a clockwise direction; wherein the second channel spirals in a counterclockwise direction, wherein, a first cubicle of the at least two cubicles is fluidly coupled to the first channel; and wherein a second cubicle of the at least two cubicles is fluidly coupled to the second channel.

In some aspects, the techniques described herein relate to a sorting track, wherein the channel includes at least one section divider.

In some aspects, the techniques described herein relate to a sorting track, further including a sorting track medium that occupies at least part of the at least two cubicles, the initial placement location, or the channel.

In some aspects, the techniques described herein relate to a sorting track, further including a guiding agent.

In some aspects, the techniques described herein relate to a sorting track, wherein the guiding agent is a nontoxic sperm attractant, progesterone, CatSper, or a calcium solution.

One aspect of this disclosure relates to separating sperm by nonlethal methods into cubicles based on their physical characteristics and/or capabilities to establish sperm categories, and then sacrificing a few members from selected ones of these cubicles to determine their genetic characteristics.

Another aspect of this disclosure relates to correlating certain category criteria with certain genetic characteristics.

Another aspect of this disclosure relates to using the correlation data to select one or more remaining sperm for fertilization from a category having one or more desired genetic traits.

In some embodiments, a sorting track for sorting living sperm into cubicles comprises: a housing; an initial placement location within the housing; a channel that is accessible to the injection area; and multiple cubicles within the housing that are each configured to be accessible from the channel; wherein the cubicles have different combinations of cubicle characteristics; wherein the cubicle characteristics include one or more of architectural characteristics and stimulus characteristics; wherein architectural characteristics include one or more of distance from initial placement location, shape of the cubicle, size or shape of a sperm-access opening into the cubicle, and cubicle side of sorting track; wherein stimulus characteristics include one or more of wavelength within the cubicle, light intensity in the cubicle, gradient of electromagnetic field in the cubicle, chemical treatment in the cubicle, and temperature in the cubicle; wherein the multiple cubicles include first and second cubicles that have a different architectural characteristic, a different stimulus characteristic, or both a different architectural characteristic and a different architectural characteristic.

In some additional, alternative, or selectively cumulative embodiments, a method for separating living sperm into categories comprises optionally exposing the sperm to an extender solution; placing sperm at an initial location in a sorting track containing multiple cubicles having different cubicle characteristics; allowing the sperm to spatially sort within the sorting track for a period of time; selecting a test sample from each of a selected number of selected cubicles, each sample including test number of sperm from a selected cubicle; categorizing the DNA from the sperm in an individual test sample into genetic characteristics; and correlating the one or more test samples with prevalent genetic characteristics.

In some additional, alternative, or selectively cumulative embodiments, a method for separating living sperm into categories comprises optionally exposing the sperm to an extender solution; placing multiple sperm at an initial location in a sorting track (or racetrack) containing multiple cubicles (or chambers or compartments) having different cubicle characteristics; allowing the sperm to spatially sort within the sorting track for a period of time; selecting a sample number of sperm from each of a selected number of selected cubicles; categorizing the sperm from each sample into identifiable characteristics including physical characteristics and/or capabilities and cubicle characteristics of the respectively selected cubicle; categorizing the DNA from the sperm in sample into genetic characteristics; correlating the one or more test samples with prevalent genetic characteristics; and correlating the genetic characteristics with the identifiable characteristics for the sperm in one or more test samples.

In some additional, alternative, or selectively cumulative embodiments, a sorting track for sorting living sperm into cubicles having openings comprises: a housing; an injection area; multiple cubicles, including first and second cubicles, that have a different combination of cubicle characteristics within the housing, wherein the cubicle characteristics include two or more of: distance from the injection area; size of cubicle opening; cubicle side of sorting track; wavelength in, or near the opening of, the cubicle; light intensity in, or near the opening of, the cubicle; gradient of electromagnetic field in, or near the opening of, the cubicle; chemical treatment in, or near the opening of, the cubicle; and temperature in, or near the opening of, the cubicle.

In some additional, alternative, or selectively cumulative embodiments, a kit for sorting living sperm into cubicles comprises: a sorting track including a housing; an initial placement location within the housing; a channel that is accessible to the injection area; multiple cubicles within the housing that are each configured to be accessible from the channel; wherein the cubicles have different combinations of cubicle characteristics; wherein the cubicle characteristics include one or more of architectural characteristics and stimulus characteristics; wherein architectural characteristics include one or more of distance from initial placement location, shape of the cubicle, size or shape of a sperm-access opening into the cubicle, and cubicle side of sorting track; wherein stimulus characteristics include one or more of wavelength within the cubicle, light intensity in the cubicle, gradient of electromagnetic field in the cubicle, chemical treatment in the cubicle, and temperature in the cubicle; wherein the multiple cubicles include first and second cubicles that have a different architectural characteristic, a different stimulus characteristic, or both a different architectural characteristic and a different architectural characteristic; a sperm collection container; and a sperm collection syringe.

In some additional, alternative, or selectively cumulative embodiments, the method comprises categorizing the sperm from each test sample into identifiable characteristics including physical characteristics and/or capabilities and cubicle characteristics of the respectively selected cubicle; and correlating the genetic characteristics with the identifiable characteristics for the sperm in one or more test samples.

In some additional, alternative, or selectively cumulative embodiments, the extender solution comprises skim milk.

In some additional, alternative, or selectively cumulative embodiments, the method further comprises separating the sperm into preliminary groups based on separate layers or genetic sexes prior to placing the sperm on the sorting track, wherein sperm from only one layer or genetic sex is placed on the sorting track.

In some additional, alternative, or selectively cumulative embodiments, a centrifuge is employed to separate the sperm into preliminary groups.

In some additional, alternative, or selectively cumulative embodiments, female genetic gender is associated with a first one of the layers and wherein male genetic gender is associated with a second one of the layers.

In some additional, alternative, or selectively cumulative embodiments, the sorting track has a generally spiral shape.

In some additional, alternative, or selectively cumulative embodiments, the initial location is a predetermined location.

In some additional, alternative, or selectively cumulative embodiments, the number of cubicles is greater than or equal to 10.

In some additional, alternative, or selectively cumulative embodiments, the number of cubicles is greater than or equal to 16.

In some additional, alternative, or selectively cumulative embodiments, the cubicle characteristics comprise distance from the initial location.

In some additional, alternative, or selectively cumulative embodiments, wherein the cubicle characteristics comprise trap opening size.

In some additional, alternative, or selectively cumulative embodiments, the cubicle characteristics comprise left side of sorting track or right side of sorting track.

In some additional, alternative, or selectively cumulative embodiments, the cubicle characteristics comprise ambient wavelength in cubicle.

In some additional, alternative, or selectively cumulative embodiments, the cubicle characteristics comprise ambient light intensity in cubicle.

In some additional, alternative, or selectively cumulative embodiments, the cubicle characteristics comprise gradient of electromagnetic field in cubicle.

In some additional, alternative, or selectively cumulative embodiments, the cubicle characteristics comprise chemical treatment associated with walls of cubicle.

In some additional, alternative, or selectively cumulative embodiments, the period of time is greater than or equal to 15 minutes.

In some additional, alternative, or selectively cumulative embodiments, the period of time is greater than or equal to 20 minutes.

In some additional, alternative, or selectively cumulative embodiments, the sample number is at least 3.

In some additional, alternative, or selectively cumulative embodiments, the selected number of selected cubicles is greater than or equal to 5.

In some additional, alternative, or selectively cumulative embodiments, the selected number of selected cubicles is greater than or equal to 8.

In some additional, alternative, or selectively cumulative embodiments, a sensor is employed to obtain data for establishing the physical characteristics and/or the capabilities.

In some additional, alternative, or selectively cumulative embodiments, an image sensor is employed to obtain data for establishing the physical characteristics and/or the capabilities.

In some additional, alternative, or selectively cumulative embodiments, a video camera is employed to obtain data for establishing the physical characteristics and/or the capabilities.

In some additional, alternative, or selectively cumulative embodiments, the physical characteristics comprise size.

In some additional, alternative, or selectively cumulative embodiments, the physical characteristics comprise shape.

In some additional, alternative, or selectively cumulative embodiments, the capabilities comprise swimming style.

In some additional, alternative, or selectively cumulative embodiments, a software processing system is employed to identify the physical characteristics and/or the capabilities from sensor data.

In some additional, alternative, or selectively cumulative embodiments, wherein the software processing system utilizes an artificial intelligence system.

In some additional, alternative, or selectively cumulative embodiments, an artificial intelligence system is employed to identify the physical characteristics and/or the capabilities from sensor data.

In some additional, alternative, or selectively cumulative embodiments, the artificial intelligence (AI) comprises one or more of a neural network, a probabilistic technique such as Bayes or Markov algorithm, a kernel method (like SVM, decision trees/random forest, Gaussians, PCA, can-cor . . . ), reinforcement learning that can have nothing to do with artificial neural networks, artificial reasoning a.k.a. “good old fashioned AI,” many path-planning and intelligent control-systems methods that correspond to “classical AI” (not the same as GOFAI), alife (swarms, cellular automata . . . ), agents and chaos systems, and/or any algorithm or group of algorithms that optimize a value function (reinforcement learning and linear dynamic programming).

In some additional, alternative, or selectively cumulative embodiments, the genetic characteristics comprise defects.

In some additional, alternative, or selectively cumulative embodiments, the genetic characteristics comprise disease susceptibility.

In some additional, alternative, or selectively cumulative embodiments, the genetic characteristics comprise genetic disorders.

In some additional, alternative, or selectively cumulative embodiments, the genetic characteristics comprise IQ.

In some additional, alternative, or selectively cumulative embodiments, the genetic characteristics comprise hair color.

In some additional, alternative, or selectively cumulative embodiments, the genetic characteristics comprise eye color.

In some additional, alternative, or selectively cumulative embodiments, the genetic characteristics comprise height.

In some additional, alternative, or selectively cumulative embodiments, an AI system is employed to correlate the genetic characteristics with the identifiable characteristics for the sperm in each sample.

In some additional, alternative, or selectively cumulative embodiments, an AI system is employed to correlate the genetic characteristics with the identifiable characteristics for each sperm in each sample.

In some additional, alternative, or selectively cumulative embodiments, the correlated characteristics are utilized to select a sperm for fertilization of an egg by choosing a cubicle and identifiable characteristics that correlate with desired genetic characteristics.

In some additional, alternative, or selectively cumulative embodiments, the sorting track controller can selectively (or entirely) vary electrical charge, magnetism, chemistry (by electrically stimulating a coated surface, by blocking a chemical reservoir), temperature, light (or various wavelengths, which can be alternated or strobed), pressure (e.g. by constricting an orifice or valve while sperm are pressed from behind or suctioned in front).

In some additional, alternative, or selectively cumulative embodiments, one or more lasers count the number of sperm in each cubicle, and an access point to the cubicle can be occluded when enough sperm have entered, causing slower sperm to sort themselves into later cubicles.

In some additional, alternative, or selectively cumulative embodiments, the number constituting enough sperm can be based on a formula that considers time since start and overall sperm count per unit volume.

In some additional, alternative, or selectively cumulative embodiments, a camera records the morphology and movement signature of a sperm before its DNA is analyzed.

In some additional, alternative, or selectively cumulative embodiments, these factors can then be used to preferentially choose a sperm from the cubicle that has the highest correlation to the one with the most desired DNA.

In some additional, alternative, or selectively cumulative embodiments, sperm capabilities, such as movement characteristics, are measured or observed under similar environmental conditions.

In some additional, alternative, or selectively cumulative embodiments, the prevalent genetic characteristics for sperm in a given cubicle are used to formulate a forecast image of a prospective person made from a sperm from the given cubicle.

In some additional, alternative, or selectively cumulative embodiments, the sperm has a sperm donor, wherein data from images of the sperm donor or a relative of the sperm donor are employed with the prevalent genetic characteristics for sperm in a given cubicle to formulate the forecast image of a prospective person made from a sperm from the given cubicle.

In some additional, alternative, or selectively cumulative embodiments, genetic characteristics of an egg donor of an egg to be used for fertilization to make the prospective person or images of the egg donor or a relative of the egg donor are employed with the prevalent genetic characteristics for sperm in a given cubicle to formulate the forecast image of the prospective person made from a sperm from the given cubicle.

In some additional, alternative, or selectively cumulative embodiments, an artificial intelligence (AI) system is employed to identify the physical characteristics or the capabilities from sensor data, is employed correlate prevalent genetic characteristics with the identifiable characteristics for the sperm in a selected sample, is employed to correlate given test samples with their prevalent genetic characteristics, is employed to formulate a forecast image of a person made from a sperm from a given cubicle.

Selectively cumulative embodiments are embodiments that include any combination of multiple embodiments that are not mutually exclusive.

Additional aspects and advantages will be apparent from the following detailed description of example embodiments, which proceeds with reference to the accompanying drawings.

### BRIEF DESCRIPTION OF THE DRAWINGS

FIG. 1 is a schematic of an embodiment of a sperm sorting track.

FIG. 2 is a pictorial view of an embodiment of a sperm sorting track, showing internal compartmentalization into cubicles.

FIG. 3 is a sectional view of an embodiment of a sperm sorting track, showing an example of a cubicle on one side of the sperm sorting track.

### DETAILED DESCRIPTION OF EMBODIMENTS

Example embodiments are described below with reference to the accompanying drawings. Unless otherwise expressly stated in the drawings, the sizes, positions, etc., of components, features, elements, etc., as well as any distances therebetween, are not necessarily to scale, and may be disproportionate and/or exaggerated for clarity.

The terminology used herein is for the purpose of describing particular example embodiments only and is not intended to be limiting. As used herein, the singular forms “a,” “an” and “the” are intended to include the plural forms as well, unless the context clearly indicates otherwise. It should be recognized that the terms “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise specified, a range of values, when recited, includes both the upper and lower limits of the range, as well as any sub-ranges therebetween. Unless indicated otherwise, terms such as “first,” “second,” etc., are only used to distinguish one element from another. For example, one element could be termed a “first element” and similarly, another element could be termed a “second element,” or vice versa. The section headings used herein are for organizational purposes only and are not to be construed as limiting the subject matter described.

Unless indicated otherwise, the terms “about,” “thereabout,” “substantially,” etc. mean that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art.

Spatially relative terms, such as “right,” left,” “below,” “beneath,” “lower,” “above,” and “upper,” and the like, may be used herein for ease of description to describe one element's or feature's relationship to another element or feature, as illustrated in the drawings. It should be recognized that the spatially relative terms are intended to encompass different orientations in addition to the orientation depicted in the figures. For example, if an object in the figures is turned over, elements described as “below” or “beneath” other elements or features would then be oriented “above” the other elements or features. Thus, the exemplary term “below” can, for example, encompass both an orientation of above and below. An object may be otherwise oriented (e.g., rotated 90 degrees or at other orientations) and the spatially relative descriptors used herein may be interpreted accordingly.

Unless clearly indicated otherwise, all connections and all operative connections may be direct or indirect. Similarly, unless clearly indicated otherwise, all connections and all operative connections may be rigid or non-rigid. As used herein, the term “fluidly coupled” is intended to communicate a connectivity that permits the passage of a fluid—liquid or gas—between the described elements in an efficacious manner for the described function of the system disclosed herein as appreciated by those of skill in the art.

Like numbers refer to like elements throughout. Thus, the same or similar numbers may be described with reference to other drawings even if they are neither mentioned nor described in the corresponding drawing. Also, even elements that are not denoted by reference numbers may be described with reference to other drawings.

Many different forms and embodiments are possible without deviating from the spirit and teachings of this disclosure and so this disclosure should not be construed as limited to the example embodiments set forth herein.

For sexual reproduction in many animal species, motile sperm must at least partially self-propel and navigate their way towards an oocyte. For example, recent studies suggest that chemotaxis, rheotaxis, and/or thermotaxis may contribute to a mammalian sperm's ability to navigate an environment towards an oocyte. A variety of genes in an organism can influence its sperm's biochemistry and morphology and therefore its sperm's ability to navigate according to chemotaxis, rheotaxis, and thermotaxis stimuli as observed in healthy samples. For example, the gene comp-1 in the worm C. elegans is not involved in spermatogenesis but is used for sperm navigation to and arrival in a spermathecac. C. elegans sperm with comp-1 knocked out are generally viable and motile but statistically fail to arrive at the spermathecae in vivo as discussed in Hansen J M, Chavez D R, Stanfield G M. COMP-1 promotes competitive advantage of nematode sperm. Elife. 2015 Mar. 19;4: e05423. doi: 10.7554/eLife.05423. PMID: 25789512; PMCID: PMC4400581.

Due to the pleiotropy of genes, it is possible that some genes governing sperm biochemistry and morphology (and therefore, their capacities for various taxes, such as chemotaxis, rheotaxis, thermotaxis, etc., as well) correlate with phenotypic conditions in somatic cells of the eventual organism, including but not limited to both disease states and disorders as well as more general appearance and behavioral traits such as hair color and measurable IQ in the larger organism. For example, sperm carrying Down syndrome often have shortened tails, influencing their motility. As another example, observed chemotaxis of sperm from the sea urchin Arbacia punctulata involves cellular Ca2+ signaling pathways. Therefore, the presence or absence of genes or alleles for genes that disrupt or otherwise modulate Ca2+ signaling elsewhere in an organism may influence the ability for its sperm to navigate by chemotaxis.

As a further example, the Tle6 gene in a knockout mice model was found to cause abnormal sperm structure in a study reported in Kazama K, Miyagoshi Y and Nishizono H (2024). Tle6 deficiency in male mice led to abnormal sperm morphology and reduced sperm motility. Front. Cell Dev. Biol. 12:1481659. doi: 10.3389/fcell.2024.1481659. More specifically, male mice heterozygous for the knockout gene were discovered to produce 57% of their sperm with an abnormal sperm head structure, possess a mating success rate and offspring count equivalent to those of wild type males, but yet demonstrate an approximately 2:1 inheritance rate of the wild type gene versus the knockout in their offspring when a 1:1 ratio would be expected by fundamental Mendelian genetics. The researchers attributed this observed deviation in expected inheritance rate to the presence of healthy sperm carrying the wild type gene that substantially out compete malformed sperm that carried the knockout. Additionally, heterozygous knockout male mice were also noted to exhibit elevated levels of testosterone compared to wild type mice. Therefore, sperm containing the knockout gene that affected sperm morphology can also be expected to cause elevated testosterone levels (and behavioral and physical changes correlating with elevated testosterone levels) in offspring produced from such sperm (e.g., through natural fertilization, in vitro fertilization, etc.).

Disclosed herein are devices and methods for sorting sperm according to differences in their motility and capacity for various taxes. A sperm sample (e.g., a sample from an individual) containing multiple individual sperm may be injected into a device containing a plurality of cubicles fluidly coupled to a channel, in some embodiments. Each cubicle possesses a unique set of cubicle characteristics as described herein that present a set of physical and/or chemical conditions to sperm traveling along the channel from the initial location. Sperm having similar morphological properties and/or capacities for different taxes will navigate themselves into corresponding cubicles. Methods involving the device may then test or sequence the genome of a portion of the sperm from each occupied cubicle to determine the presence or absence of one or more genes correlating with one or more traits (e.g., a disease state, a disorder, an appearance such as hair color, hair texture, skin color, and eye color, or a measurable IQ, etc.) statistically representative of the population of sperm within the cubicle, in some embodiments. In some embodiments, cameras located inside each cubicle may alternatively, or additionally, photograph the morphological characteristics of the population of sperm within the cubicle. Remaining sperm in a cubicle containing desirable traits may then be selected for further purposes, including, but not limited to, fertilization of an oocyte. Although much of the language used herein is directed towards a sorting of sperm, the devices and methods herein may be used to sort other motile, microscopic entities as well, in some embodiments, including, but not limited to bacteria, archaca, protozoa, viruses, spores, mitochondria, red blood cells, white blood cells, and platelets.

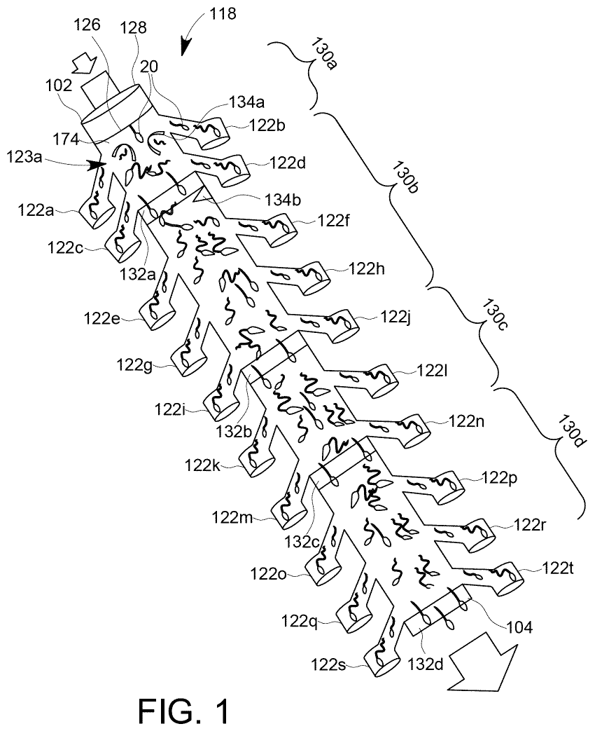

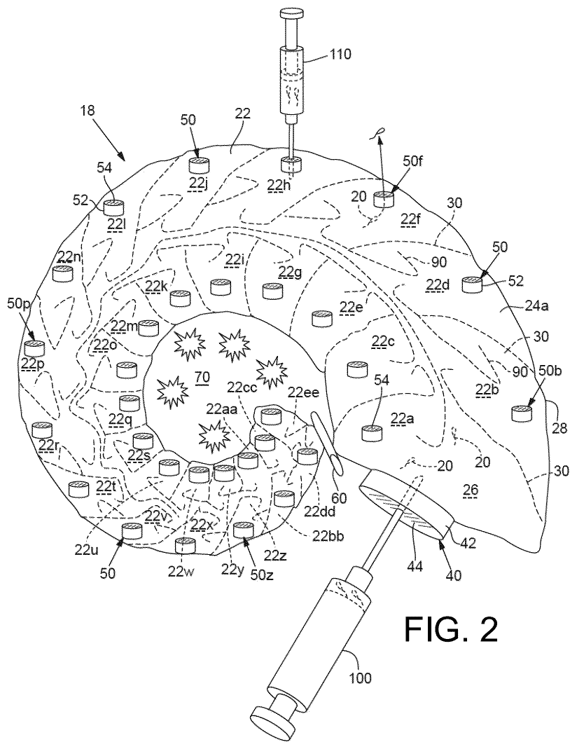

FIG. 1 shows a schematic (features not to scale) for an embodiment of a sperm sorting track 118 that is configured to sort sperm 20 into multiple different cubicles 122, (e.g., at least two cubicles 122), such as cubicles 122a, 122b, 122c, 122d, 122e, 122f, 122g, 122h, 122i, 122j, 122k, 122l, 122m, 122n, 122o, 122p, 122q, 122r, 122s, and 122t, fluidly coupled to a channel 174 by at least one entrance 123 (e.g., entrance 123a) as defined by a housing 128 (e.g., an outer shell). Cubicles 122 may have any arrangement around the channel 174, including but not limited to, arranged on a left and/or right side and a radial arrangement in some embodiments. Cubicles 122 may be spaced evenly on unevenly from each other, in some embodiments. In some embodiments, sperm 20 placed into an initial placement location 126 near a first end 102 of the sorting track 118 may travel along the channel 174 towards a second end 104 and enter a cubicle 122 according to their motility and/or capacity for different taxes as accommodated or attracted by a cubicle's 122 unique set of cubicle characteristics. In some embodiments, the cubicle characteristics include one or more architectural characteristics including, but not limited to, a distance from the initial placement location 126, a cubicle shape, a cubicle volume, a cubicle position within the housing 128 relative to the channel 174, an entrance size, and an entrance shape. In some embodiments, the cubicle characteristics may additionally include one or more stimulus characteristics including, but not limited to, a wavelength of light, a light intensity, an electromagnetic field, a radioactive radiation intensity, a chemical treatment, a pressure, a sound, and a temperature. One or more cubicles 122 may include at least one extraction portal (e.g., extraction portal 50 of FIG. 2).

Although shown as a linear path in the embodiment of FIG. 1, the channel 174 may have any shape, including, but not limited to, a generally spiral shape in either a clockwise or counterclockwise direction, a chevron shape, a slanted shape, etc. In some embodiments, the channel 174 and corresponding cubicles 122 may divided into two or more sections 130 such as sections 130a, 130b, 130c, and 130d. In some embodiments, two sections may be separated by a section divider 132 such as section dividers 132a, 132b, 132c, and 132d. A section divider 132 may include a porous membrane, in some embodiments, such as a filter or cheese cloth with or without an additional chemical coating. In some embodiments, the section divider 132 may emulate the physical and/or chemical conditions required for sperm 20 to fertilize an egg cell. In some embodiments involving multiple section dividers 132, each section divider 132 may be identical. In some embodiments involving multiple section dividers 132, cach section divider 132 may be different. In some embodiments involving multiple section dividers 132, cach section divider 132 nearer the second end 104 of the sorting track 118 may be progressively denser than the previous section divider 132. When a sperm 20 passes through section divider 132d of FIG. 1, the sperm may enter another section (not shown) of the sorting track 118, in some embodiments, or it may exit the sorting track 118 for future operations or testing.

In some embodiments, cubicles 122 of a section 130 may share one cubicle characteristic (c.g., a stimulus characteristic) while differing in at least another (c.g., an architectural characteristic, such as a distance from the initial placement location 126 and/or a cubicle position within the housing 128 relative to the channel 174). For example, cubicles 122a, 122b, 122c, and 122d of section 130a may include positive or negative electrodes present in their cubicle volumes or at or near their correspondence entrance 123 to produce an electromagnetic field, static or cycling. In some embodiments, cubicles 122a, 122b, 122c, and 122d may produce an identical electromagnetic field and therefore share a stimulus characteristic while remaining differentiated by their different architectural characteristics of their distances from the initial placement location 126 and a position within the housing 128 relative to the channel 174. In at least this manner, cubicles 122a, 122b, 122c, and 122d cach exhibit a unique set of cubicle characteristics despite sharing a stimulus characteristic. In some embodiments, cubicles 122a, 122b, 122c, and 122d may produce electromagnetic fields having different parameters (c.g., strengths, polarities, cycling patterns, etc.) and therefore are further differentiated from each other by their stimulus characteristics. Sperm 20 responsive to these characteristics (e.g., position along the channel 174, electromagnetic strength, etc.) may navigate themselves into cubicles 122a, 122b, 122c, and 122d, in some embodiments, or otherwise may be repelled and navigate to another cubicle 122. In some embodiments, cubicles 122 (e.g., cubicles 122a, 122b, 122c, and 122d) of a first section 130a nearest the initial placement location 126 include no stimulus characteristic, therefore allowing the sperm 20 to sort themselves substantially or entirely by motility properties within the first section 130a.

As further examples, cubicles 122e, 122f, 122g, 122h, 122i, and 122j of section 130b may produce different intensities and/or wavelengths of light, cubicles 122k, 122l, 122m, and 122n of section 130c may produce different intensities and wavelengths of pressure or sound, and cubicles 122o, 122p, 122q, 122r, 122s, and 122t of section 130d may produce different temperatures. In some embodiments, the cubicles 122 of a section 130 (e.g., cubicles 122a, 122b, 122c, and 122d of section 130a) do not share a stimulus characteristic. For example, cubicle 122a may produce an electromagnetic field and include no chemical treatment, and cubicle 122b may include a chemical treatment and produce no electromagnetic field.

Sperm 20 responsive to or compatible with the cubicle characteristics of the cubicles 122 may sort themselves accordingly along the length of the channel 174 of the sorting track 118. For example, sperm 20 with short tails that limit their motility may only be found in one or more cubicles 122 nearer to the initial placement location 126 (e.g., an architectural characteristic) than sperm 20 with healthy tails. Furthermore, for example, sperm 20 with mishappen heads may drift in a particular direction while trying to navigate the towards the second end 104 of the channel 174 (c.g., as motivated by rheotaxis or chemotaxis forces provided by the sorting track 118, sorting track medium, and/or guiding agent as described herein in some embodiments). The sperm 20 in this example may therefore sort themselves into cubicles 122 having the architectural characteristic of being located on a particular side of the channel 174. As another example, sperm 20 attracted to a chemical treatment (e.g., a stimulus characteristic) of a cubicle 122 may be drawn into that cubicle 122.

Combinations of cubicle characteristics may influence self-sorting of a sperm 20 in a sorting track 118 in some embodiments. For example, a sperm 20 particularly susceptible to a particular chemical treatment (e.g., a sperm attractant) may nonetheless also possess morphological characteristics that produce a drift in its motion. Therefore, sperm 20 of this example may preferentially sort themselves into a cubicle 122 (e.g., cubicle 122a) having a stimulus characteristic of the sperm attractant chemical treatment and the architectural characteristic of having a certain position relative to the channel 174 as opposed to another cubicle 122 (c.g., cubicle 122b) having the same chemical treatment but a different position relative to the channel 174.

Because sperm morphology and capacity for response to various taxes can correlate with the carried haploid genetic payload of a given sperm, sperm 20 that sort themselves into a specific cubicle 122 are likely to share at least some genetic similarities in their genetic payloads. For example, sperm 20 that arrived in a particular cubicle 122 due to a shorter tail influencing their motility in a specific pattern may likely share one or genes in their genetic payload that result in a shorter tail. These one or more genes resulting in a shorter tail may correlate with broader traits in the organism as a whole, such as with the malformed sperm head and heightened testosterone levels in mice in the study described above. As described herein, at least some of the sperm 20 within a cubicle 122 after a sort may be extracted using an extraction portal (e.g., extraction portal 50 of FIG. 2) and tested for one or more specific genes. In some embodiments, sperm 20 within a cubicle may be photographed externally after extraction or within the sorting track 118 by one or more cameras or image sensors located within a cubicle 122.

In order to aid in a more equal exposure of sperm 20 to the plurality of cubicles 122 the channel 174 may feature one or more mixing features 134 (e.g., mixing features 134a and 134b) in some embodiments. In some embodiments, mixing features 134 may include, but are not limited to a static baffle, a perturbance from the housing 128 into the channel 174, or a motorized stirrer. For example, the initial placement location 126 and/or a portion of the channel 174 near the initial placement location 126 may include one or more mixing features 134a. In some embodiments, one or more mixing features 134b may be located elsewhere in the channel 174 including, but not limited to, before and/or after a section divider 132.

FIG. 2 is a pictorial view of an embodiment of a sperm sorting track 18 that is configured to sort sperm 20 into multiple compartments or cubicles 22, such as cubicles 22a, 22b, 22c, 22d, 22c, 22f, 22g, 22h, 22i, 22j, 22k, 22l, 22m, 22n, 220, 22p, 22q, 22r, 22s, 22t, 22u, 22v, 22w, 22x, 22y, 22z, 22aa, 22bb, 22cc, 22dd, and 22cc, (c.g., at least two cubicles 22) that have different characteristics. Sperm 20 placed into an initial placement location 26 may travel down a channel (e.g., channel 174 of FIG. 1) and enter a cubicle 22 according to various properties of a given sperm cell's morphology, motility, and capacity for various taxes (c.g., rheotaxis, chemotaxis, thermotaxis, etc.) A test sample, including a test number of sperm 20, can be obtained from each of selected ones of the cubicles 22. The DNA of the sperm 20 in the selected test samples can be analyzed, and the sperm 20 from the selected test samples can be categorized by their prevalent genetic characteristics and correlated with the respectively selected cubicle 22 (and optionally its cubicle characteristics). Optionally, the sperm 20 from each test sample can also be categorized into identifiable characteristics including physical characteristics and/or capabilities. The DNA of the sperm 20 in the selected test samples can be analyzed, and the identifiable characteristics for the sperm 20 in each test sample can be correlated with prevalent genetic characteristics.

The sorting track 18 may have an exterior shape like that of a conch shell, such as shown in FIG. 2, or it may have a shape more similar to a spiral staircase. However, a variety of other shapes may be substituted. The spiral direction (or Coriolis direction) can be counterclockwise as shown or clockwise (not shown). The spiral can be partly, mostly, or all in the same plane or the spiral may gradually or precipitously change elevation (as a consistent change or and inconsistent change) with respect to the elevation of an initial placement location 26. The spiral may decrease in size (such as the size of its cross-sectional area) from its beginning to end, such as shown in FIG. 2, or the spiral may have a substantially uniform cross-section from its beginning to end. The change in size may be gradual and/or consistent, or the change may be precipitous and/or inconsistent.

In some embodiments, the sorting track 18 may have the appearance of twine, with two pathways, such as a first channel and a second channel, that twist around each other with cach channel presenting a different Coriolis direction with the initial placement location 26 positioned in proximity to a junction that separates the two pathways. This type of sorting track 18 may be formed in a spiral as a whole or may be linear. In some embodiments a first cubicle of the at least two cubicles 22 may be fluidly coupled to the first channel while a second cubicle of the at least two cubicles 22 may be fluidly coupled to the second channel. The cubicles 22 may have the same or different sizes. The initial placement location 26 may be near the beginning of the twine pathways or partway downstream of the beginning. In some embodiments, the two pathways may be completely separated, cach with its own entry portal 40, but attached to save space in a 3D printer of for packing and shipping. These pathways could alternatively not be attached at all.

The sorting track 18 and its cubicles 22 may be constructed of a material that is not harmful to sperm, 20 either by leaching out into the extender, by radiation, or by contact. The sorting track 18 may be made of a material, such as glass or plastic. However, any suitable material or any combination of suitable materials can be employed. For examples, some suitable materials include FDA-approved materials for use inside the human body, such as Noryl™. Other suitable materials might include materials that can be utilized in 3D printers, such as materials used in fused deposition modeling (FDM), digital light processing (DLP), or stereolithography (SLA) printers. Conventional consumer FDM printers can provide a layer thickness of 0.2 or 0.3 mm. Some better desktop models even claim a vertical resolution of 0.02 mm. Readily available SLA printers such can provide layers as thin as 0.025 mm. More advanced SLA printers can offer a 25-micron XY resolution and 25-300 microns (user selectable) in the Z, using an 85-micron laser. The actual accuracy may depend on many factors such as the print performance of an individual 3D printing resin.

While 3D printing is an example of one manufacturing method, any suitable manufacturing method may be employed. Other manufacturing techniques may include injection molding or casting. These techniques may form the sperm sorting track 18 as single production piece, or these techniques may be employed to produce the sperm sorting track 18 as two or more pieces that are subsequently connected.

The sorting track 18 may include a housing, such as an outer shell 28, and inner barriers 30 that form the cubicles 22 fluidly connected to a channel (e.g., channel 174 of FIG. 1). In at least this manner, the housing may define at least two cubicles 22, in some embodiments. In some embodiments, the housing may define two cubicles to ten cubicles 22. In some embodiments, the housing may define two cubicles 22 to one hundred cubicles 22. The outer shell 18 may have an identifiable entry portal 40 that may be located near the initial placement location 26. The entry-portal 40 and/or the initial placement location 26 may be positioned near the largest portion of the sorting track 18, such as in proximity to the largest cubicles 22, if they differ in size. In at least this manner, a housing (e.g., outer shell 28) may define the initial placement location 26. In other embodiments, the entry-portal 40 and/or the initial placement location 26 may be positioned up to about 15% downstream from the largest cubicles 22, such as near cubicles 22e and 22d in FIG. 2 with a junction that would allow sperm 20 to sort to either side 24 of the sorting track 18. In some embodiments, the channel is fluidly coupled to the cubicles 22 (e.g., at least two cubicles), and the initial placement location 26.

The entry portal 40 may include an entry lip 42 that supports an injection area 44. The outer shell 28, inner barriers 30, entry lip 42, and injection area may be formed of the same material(s) of they may be formed of one or more different materials. They may be 3D printed as an integrated unit or they may be manufactured separately and assembled together. In some embodiments, the injection area 44 may employ a thin self-sealing material, such as a rubber-like material.

The outer shell 28 may also include an extraction portal 50 for each of the cubicles 22. The extraction portals 50, such as extraction portals 50a, 50b, 50c, 50d, 50e, 50f, 50g, 50h, 50i, 50j, 50k, 50l, 50m, 50n, 50o, 50p, 50q, 50r, 50s, 50t, 50u, 50v, 50w, 50x, 50y, 50z, 50aa, 50bb, 50cc, 50dd, and 50cc, may each include an extraction lip 52 and an extraction area 54. The extraction portals 50 may include the same materials as those used to form the entry portal 40.

The injection portal 40 and the extractions portals 50 may all have the same size and shape, or the injection portal and the extraction portals may have different sizes and/or different shapes. For example, the injection portal 40 and the extractions portals 50 may both be generally configured as circles, disks, or cylinders as shown; however, the injection portal 40 may have a larger size to accommodate a bigger syringe bore, and the extractions portals 50 may have smaller sizes because the cubicles may diminish in size. In some embodiments, the major dimension (or diameter) of the injection portal 40 may be less than or equal to 1 inch or less than or equal to ½ inch. In some embodiments, the major dimension (or diameter) of the extraction portal 50 may be less than or equal to ½ inch or less than or equal to ¼ inch.

The sorting track 18 may also include a central access portal 60, defined by the housing (c.g., outer shell 28) in some embodiments, to provide a pathway for depositing central guiding agent compositions into a central compartment 70 toward an imaginary central axis of the spiral. The central access portal 60 may be configured like the injection portal 40 and the extractions portals 50, or it may be configured differently. For example, the central access portal 60 may employ a tube-like structure that has an injection area that is smaller than the injection area 44 and the extraction area 54. In some embodiments, the central access portal 60 may be located on a second end (c.g., second end 104 of FIG. 1) of a channel (c.g., channel 174 of FIG. 1) wherein a first end of the channel is fluidly coupled to the initial placement location 26. The central guiding agent may include one or more nontoxic sperm attractants. Progesterone, CatSper, and various calcium compounds, in suspension, with a concentration somewhat less than saturated, can be used to excite the tails of sperm 20 to flagellate excitedly. These substances tend to aid in penetrating an egg, but they can also act as sperm attractants as they may aid sperm 20 to pass through a porous barrier into a compartment or cubicle 22, and/or these substances may be employ to change the original pattern or direction of movement of the sperm 20 to aiding in the sorting process.

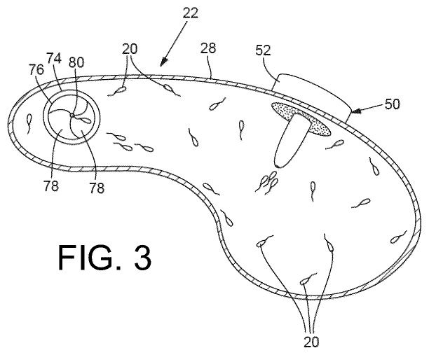

FIG. 3 is a sectional view of an embodiment of the sperm sorting track 18, showing an example of a cubicle 22, such as the cubicle 22d on one side 24a of the sperm sorting track 18. (The cubicle shown in FIG. 3 is not an exact cross-section of the sorting track 18 shown in FIG. 2.) The number of cubicles may range from 2 to 100. The number of cubicles 22 may be greater than or equal to 5 or greater than or equal to 8. However, the sorting track 18 may generally include 10 or more cubicles 22 and fewer than 50 cubicles 22, or more than 16 cubicles 22 and fewer than 50 cubicles 22. In some embodiments, the sorting track 18 may have greater than or equal to 20 cubicles 22 and less than or equal to 40 cubicles 22. One will appreciate, however, that the number of cubicles 22 could be greater than or equal to 100.

Each cubicle is generally large enough to hold enough sperm 20 and an extender to be useful for transferring enough of the mixture into a straw for freezing and later insemination. The extender solution may be dynamically refreshed to each cubicle 22 if too much flows back out of an opening.

The cubicles 22 may be directly or indirectly accessible from the initial placement location 26, such as through a channel 74 that may be generally located along a midline of the curvature of the sorting track 18. In some embodiments, the housing (e.g., outer shell 28) may define the channel 74. In some embodiments, the channel 74 may include a plurality of channels (c.g., a first channel and a second channel) that may run substantially parallel and adjacent to each other or in another direction. In some embodiments, the channel has a generally spiral shape. The entrances to these cubicles 22, which fluidly couple cubicles 22 to the channel 74, in some embodiments, may be just larger than a sperm 20 in order to facilitate quick closing when the required quantity is reached, or the entrances may be larger, or much larger. The entrances may include valves 76 or other features to partly limit passage of sperm between cubicles 22. The valves 76 may include one or more flaps 78 that may, or may not, partly overlap. The valves 76 may be connected by tube-like structures that have multiple openings that permit sperm to escape, or the valves 76 may be completely unobstructed with respect to the other areas of the cubicles 22.

The valves 76 may have a major dimension (such as a diameter) that may range from about 1/32 inch to about ½ inch, or 1/16 inch to ⅜ inch, or ⅛ inch to 5/16. In some embodiments, the valves 76 may have a major dimension that is greater than or equal to ¼ inch. One will appreciate, however, that the valve major dimension may be shorter than 1/32 inch or may be longer than ½ inch. One will also appreciate that while the valves 76 may have a circular shape, other suitable shapes can be employed. Additionally, the valves 76 may all be the same size and shape, or they may have different shapes and/or sizes. For example, the valves 76 may diminish in size as the cubicles 22 diminish in size.

The flaps 78 may limit the size of the aperture 80 through the valves 76 (and through the entrance, such as entrance 123a of FIG. 1). In general, the aperture 80 may have a major dimension, such as a diameter, that is greater than or equal to about 5 microns. (The human sperm cell is about 3-5 um wide at its widest part, so it should be able to fit through any gap that size or larger. Horse sperm heads are about the same, with one study showing an average width of 2.89 micron.) Moreover, the aperture 80 may have a major dimension (such as a diameter) that may range from about 3 microns to about 4 microns, about 4 microns to about 5 microns, about 5 microns to about 12.7 mm, or about 50 microns to about 10 mm, or about 100 microns to about 8 mm. In some embodiments, the aperture 80 may have a major dimension that is greater than or equal to 10 microns. One will appreciate, however, that the aperture major dimension may be shorter than 10 microns or may be longer than 12.7 mm. One will also appreciate that while the aperture may have a circular shape, other suitable shapes can be employed.

Additionally, the apertures 80 may all be the same size and shape, or they may have different shapes and/or sizes. For example, the apertures 80 may diminish in size as the cubicles 22 or valves 76 diminish in size. Alternatively, if a cubicle 22 is smaller, by its nature of being further along the path of the “shell”, the cubicle 22 may have an entrance or aperture 80 that is larger, may stay open longer, and/or may have more attractants for sperm 20. Spatially carlier cubicles 22 may be configured to be more discriminating, with smaller, harder to access entrances or apertures 80, and these earlier cubicles may employ multiple methods to repel sperm from those entrances or apertures 80.

Early cubicles 22 may also offer barriers, doors, or valves that are configured sort out left-handed and right-handed helix swimming sperm 20. One will appreciate that only about 4-5% of the motile human sperm 20 swim along well-defined helices. Furthermore, among these observed helical human sperms, a significant majority (approximately 90%) preferred right-handed helices over left-handed ones, with a helix radius of approximately 0.5-3 μm, a helical rotation speed of approximately 3-20 rotations/s and a linear speed of approximately 20-100 μm/s. The compartment, cubicle 22, or sub-cubicle access points can be configured to differentiate sperm 20 based on different helical-swimming patterns beyond handedness. For example, the planes of the access points may be angled in different direction and/or at different angles to a channel. In some embodiments, an entrance (e.g., 123a of FIG. 1) of a cubicle 22 may further comprise an entrance baffle. In some embodiments, the entrance baffle positioned on the channel 74 side of an entrance to a cubicle 22 may disrupt a flow of sperm 20 through the channel 74 to ensure sperm 20 are given sufficient time to be exposed to any stimulus characteristics of a cubicle 22 as described herein. In some embodiments, the entrance baffle positioned on the channel 74 side of an entrance to a cubicle 22 may disrupt a flow of sperm 20 through the channel 74 to direct an approximately equal count of sperm 20 into each cubicle 22.

With reference again to FIG. 3, the cubicle 22 may have a cross-sectional shape like that of kidney bean, as shown; or the cubicle 22 may have a cross-sectional shape like that of a circle, ellipse, oval, rectangle, square, hexagon, pentagon, other polygon, or other suitable shape. In some embodiments, the cubicles 22 may resemble a mini lobster trap (c.g., having an at least partially conical or frustoconical shape with a narrower diameter end and a wider diameter end) or maze (e.g., maze-shaped), where the sperm can swim in, but not back out. The cubicles 22 may all have the same shapes and dimensions; or, the shapes and/or dimensions of the cubicles 22 may be different. For example, the cubicles 22 may diminish in volume from the initial placement location 26 to the central compartment 70. Moreover, in embodiments that are substantially bilateral, the cubicles 22 may have substantially mirror image shapes, or the compartments or cubicles 22 on the inner side of the spiral may be smaller.

Each cubicle 22 may have a generally uniform interior width (along the direction of the channel 74). However, the width may be smaller or larger near the channel 74, or the width may taper in a way that is either smaller or larger toward the channel 74. The portion of the cubicle 22 that supports the extraction portal 50 will generally have a width at least as big as the diameter of the extraction portal 50, such as greater than or equal to ½ inch.

In some embodiments, the cubicles 22 may have width (along the channel), depth (in a dimension perpendicular to the width and a direction extending outwardly from the channel 74), and height (perpendicular to the width and depth) dimensions that at less than or equal to 5 inches. In particular, the width may range from about 5 inches to about ½ inch, the depth may range from about 5 inches to about ½ inch, and height may about 4 inches to about ½ inch. One will appreciate that any of the dimensions can be smaller than ½ inch or greater than 5 inches. In some examples, larger cubicles 22 may have a width that is greater than or equal to 3.5 inches and may have a depth (in a dimension perpendicular to the width and a direction extending outwardly from the channel 74) that is greater than 1 inch; and the cubicles 22 may become progressively smaller until they are less than or equal to 0.5 inch deep and 1 inch wide.

Typically, the location of the channel 74 or the valve 76 will be asymmetrically displaced with respect to the cross-sectional shape of the cubicle 22. However, one will appreciate that the channel 74 or the valve 76 may be located as centrally as practical within the cross-sectional shape of the cubicle 22. Additionally, one will appreciate that the channel 74 or the valve 76 may be located in generally the same respective location in each cubicle (on the same side of the sperm sorting track 18) or they may have different respective locations.

The cubicles 22 may also include inner obstacles 90 that may help to trap sperm within a cubicle 22. The inner obstacles 90 may extend the full height of the cubicle 22 or may block only a portion of the cubicle 22. For example, inner obstacles 90 may include funnels, paddle wheels, or valves not unlike those in blood vessels. In some embodiments, these obstacles 90 may be coated with chemicals, electrified, piezoelectrically activated (such as to bend or vibrate), or heated.

Sperm 20 swim through a fluid by a periodic wave-like beating of their flagellum. Studies show how sperm 20 congregate in a given wave scenario. At low Reynolds numbers and in confinement, the directed motion of sperm 20 is strongly influenced by steric and hydrodynamic wall interactions. Changes in the sidewall modulations and the imposed beat pattern allow the identification of a strong dependence of the surface attraction on the beat-shape envelope of the sperm 20. Detachment of sperm, while swimming along curved walls, is dominated by the change of beat-plane orientation. Therefore, either the emergence of a nonplanar component of the flagellar beat with increasing wavelength or the strong confinement in shallow channels drastically increases wall attraction. See “Sperm motility in modulated microchannels,” Rode, Sebastian et al. New J. Phys. 21 013016 (18 Jan. 2019). Accordingly, piezoelectrically activated walls (within or outside of the cubicles 22, e.g., in the channel 74) or piezoelectrically activated inner obstacles 90 may be employed to modify the oscillations in the channels of the sorting track 18 by varying the wave characteristics, such as by ramping up and down, can herd the receptive sperm 20 toward various cubicles 22 or sub-cubicles. In at least this manner, activating piezoelectrically activated walls may be said to vary a pressure within the channel 74 or within a cubicle 22 as a stimulus characteristic.

Accordingly, the cubicles 22 may also be substantially filled with a sorting track medium within which the sperm 20 can swim. In some embodiments, the sorting track medium may occupy at least part of the cubicles 22, the initial placement location 26, or the channel 74. For example, this medium may include one or more of a) a physical porous material that acts as a size sorting grid, b) a chemical or suspended mineral that energizes or attracts sperm (e.g., sperm attractants), such as progesterone, CatSper, calcium compounds in suspension, albumin, salt, sugar acid, base, oils, or c) a chemical or suspended mineral that repels sperm (e.g., a sperm repellant), such as synthetic Progesterone Receptor Ligands (sPRL, known as contraceptives) and zinc (a cation released by the oocyte upon fertilization). In order to effectively repel mammalian sperm 20, they made need to be capacitated first.

The cubicles 22 are configured to have different cubicle characteristics. A cubicle characteristic can be any aspect of the cubicle that might differentiate it from another cubicle 22. In at least this manner, a cubicle 22 may include a unique set of cubicle characteristics. A cubicle characteristic may include an architectural feature of the cubicle 22. For example, an architectural characteristic may include distance (along the channel 74) of the cubicle 22 from the initial placement location 26 and/or the side 24 of the sorting track 18. Another architectural characteristic may include the shape and/or major dimension of a sperm access opening into the cubicle 22, such as aperture 80 of the channel 74 at the entrance and/or the exit of the cubicle 22 (if the apertures 80 are a variable). One will appreciate that cubicles 22 may, for example, be accessible to the channel 74 through other cubicles 22 or through minor channels that branch off the channel 74. The shape and/or size of the cubicle cross section and/or the configuration of any inner obstacles 90 may also be an architectural characteristic. In some embodiments, architectural characteristics may include, but are not limited to, a distance from the initial placement location, a cubicle shape, a cubicle volume, a cubicle position within the housing relative to the channel, an entrance size, and an entrance shape.

A cubicle characteristic may also include a sperm stimulus such as any attribute that might attract or repel an individual sperm. For example, a stimulus characteristic might include light, temperature, magnetic field strength, or other condition. In particular, a stimulus characteristic may also include the nature of any light within a cubicle 22 including the intensity or wavelength of light within a cubicle 22. A cubicle 22 may be constructed so that the material of its outer shell 28 may be configured to allow entry of ambient light of different intensities and/or that may be configured to filter and deny or propagate only selected wavelengths. In some embodiments, a stimulus characteristic may include, but is not limited to, a wavelength of light, a light intensity, an electromagnetic field, a chemical treatment, a pressure, and a temperature. In some embodiments, a stimulus characteristic may be present in a cubicle 22 or at or near the entrance of the cubicle 22. For example, a cubicle 22 may be constructed so that its outer shell 28 may allow one or more wavelengths of light to enter. For example, a cubicle 22 may include an LED positioned near its entrance on either the cubicle side or the channel 74 side of the entrance.

Alternatively, construction of a cubicle 22 may include fabrication of LEDs or other light sources within the walls (outer shell 28 or inner barrier 30) of the cubicle 22. One will appreciate that light source and circuit elements can be 3D printed; or these elements can be later installed into prefabricated half sections of the sorting track 18 that can be subsequently assembled. These light sources may be connected to through printed and/or added circuitry to a power port configured to mate with a power cord that is configures to mate with a power source, such as a wall outlet or a USB port of a computer.

Similarly, a cubicle characteristic may include a gradient of an electromagnetic field. For example, the outer shell 28 of the cubicles 22 can be configured to include different coil spacing of wiring which can vary field strength. An example of field strengths may be from 1-8 gauss varied at 15-50 Hz. Alternatively or additionally, a cubicle characteristic may also include temperature. Temperature variation between cubicles may also be provided by internal wiring or may be a result of different wall thicknesses or other wall properties in response to ambient temperature.

A cubicle characteristic may include a chemical treatment. In some embodiments, the chemical treatment may include one or more chemicals that are employed to treat or attach to the interior walls of the cubicles 22 and or the chemical treatment may include one or more chemicals that are injected into the cubicles 22 through the extraction portal 50. These chemicals are preferably not harmful to the sperm or the DNA that the sperm contain. Some chemicals may, however, be implemented as gel barrier that may prevent some sperm 20 from accessing some of the cubicles 22. Some of these chemicals may include sperm attractants, as previously described. In some embodiments, the chemicals may include sperm repellants, as described herein. These chemicals may be provided in relatively low doses, such as about 100 micrograms per milliliter. One will appreciate that the actual concentration may depend on a number of variables as well as the specific substance.

The sorting track 18 and cubicles 22 may also provide cubicle characteristics configured to sort based on genetically-based differences in the rate and intensity of cytoplasmic and/or voltage-sensitive flickering; the number, density, and/or size of acrosomal clusters, and their sperm-region location bias (within a given region, the size of each region overall, and whether or not activity (magnitude/frequency is above a threshold in a region). See “Patch-clamp ‘mapping’ of ion channel activity in human sperm reveals regionalisation and co-localisation into mixed clusters,” Jiménez-González, M. C. et al., J Cell Physiol. 2007 December; 213(3): 801-808.

Typically, cach cubicle 22 may have three or more cubicle characteristics, including the cubicle distance and cubicle side 24. In some embodiments, cach cubicle 22 may have four or more cubicle characteristics. One will appreciate that some of the cubicles 22 may share some cubicle characteristics. For example, two cubicles 22 that are the same or similar distances from initial sperm-placement 26 location but are located on different sides 24 of the sorting track 18 may share the remaining set of cubicle characteristics. For example, one cubicle 22 may be defined by cubicle distance, cubicle side 24, and cubicle temperature. Another cubicle may be defined by cubicle distance, cubicle side 24, and cubicle light. Another cubicle may be defined by cubicle distance, cubicle side 24, and cubicle temperature, and magnetic field strength. One will appreciate that many combinations of the above described cubicle characteristics are possible.

In one embodiment, a series of cubicle doors may be aligned along a spiral around the tunnel configured for sperm 20 travel. At different cubicle entrances, LEDs flash different colors, and piezo electrically powered paddles set up different standing waves for the sperm 20 to have to change their flagellation frequencies to pass through. One side of the channel may be heated, or one side of an inner obstacle may be heated. Behind the sperm 20, either in the channel or within a cubicle 22, a permeable wall may emit a chemical that attracts or repels the sperm 20 either toward or away from a cubicle 22 or sub-cubicle. Roughly perpendicular and adjacent to the heated wall may be an electromagnetically variable surface that increases and/or decreases the gauss (magnetic flux density or magnetic induction) to which the sperm 20 are exposed.

In some embodiments, a sorting track controller can selectively (or entirely) vary some of the cubicle characteristics before or during a sperm sort. In some cases, this variation ability can be employed to calibrate some characteristics to account for differences in sorting track manufacture or to account for predetermined characteristics of the sperm donor, such as age, weight, ancestry, etc. These variable cubicle characteristics may include one or more of electrical charge, magnetism, chemistry (by electrically stimulating a coated surface, by blocking access to a cubicle or a sub-cubicle, or by providing a chemical reservoir for some other purpose), temperature, light (or various wavelengths, which can be alternated or strobed), pressure (e.g. by constricting an orifice or valve while sperm are pressed from behind or suctioned in front). For example, a sorting track controller may vary a pressure produced by a piezoelectric component of the channel 74 or cubicle 22 in order to generate pressure waves, sound waves, or a pressure gradient capable of stimulating a rheotaxis response in sperm 20. These characteristics can also be changed in response to sensor data obtained during the sort. For example, chemicals may be released to address slow swimming speed or an electrical signal may be sent to block access to a cubicle 22 that has obtained more than a certain number of sperm 20. In particular, Lily of the Valley cardiac glycosides, convallotoxin, convallatoxol, convalloside, convallatoxoloside, convallarin, glucoconvalloside, and/or convallamarin (as well as previously mentioned sperm attractants may be released to excite flagellation in the sperm 20; and 1 gauss at 50 Hz will inhibit motility in sperm.

There are numerous ways to block the path of the sperm 20. Some examples employ: a semipermeable membrane that sperm 20 need to wiggle through; a grid that only allows a smaller diameter of sperm through; a valve that allows forward progress, but no retreat; a paddle wheel that only opens if enough sperm press at the same time; a valve that opens and closes at a certain frequency; and a surface that is treated with a chemical or other substance that attracts or repels sperm 20 to varying degrees. Moreover, the pathway or entrance can be “blocked” with an attractant on a pinwheel that can lure sperm 20 into a cubicle 22, or the pathway or entrance can be “blocked” with a repellant baffle that redirects sperm 20 away to another cubicle. Repellant baffles may be considered an architectural characteristic of an entrance shape.

If a large quantity of sperm 20 manage to gather in the central compartment 70, these sperm 20 may be placed into another sorting track 18 that may be the same or different than that provided for the first sorting track 18.

In one example of a typical process, a man provides a sperm donation. Even sperm cells from the same man will exhibit large genetic differences. See “It DOES make a difference which wins the race: Scientists scan 100 sperm from one man-and find huge DNA differences” https://www.dailymail.co.uk/sciencetech/article-2176016/It-DOES-make-difference-wins-race-Scientists-scan-100-sperm-man--huge-DNA-differences.html.

This sperm donation can be collected by ejaculation into a container, such as a cup or other vessel, or by a surgical or other method. The sperm 20 may be mixed with one or more nontoxic additives that may extend sperm vitality. One example of a sperm additive includes a skim milk solution, which is typically used as an extender for shipping or storing semen. Other examples include egg yolk, coconut milk, and soya milk, individually or in combination. In particular, a syringe with skim milk solution may be employed to rinse the container, which is then swirled to mix with the semen, and then the combined solution is drawn back up into the syringe.

In an example of an optional preliminary sorting process to sort the sperm 20 into preliminary groups, such as a gender sorting process, the combined solution including the sperm 20 and extender, may be placed (or injected) into a centrifuge test tube. Through a gentle spin cycle in a centrifuge, the heavier sperm 20 (sperm bearing an X-chromosome, also called X-sperm or female sperm 20) forms a bottom layer within the test tube and the lighter sperm 20 (sperm bearing a Y-chromosome, also called Y-sperm or male sperm 20) forms a top layer that may be distinguishable from the bottom layer. A mini syringe 100 can be used to extract a suitable number of female sperm 20 from the bottom sperm layer of the test tube, and/or a mini syringe 100 can be used to extract a suitable number of or male sperm 20 from the top sperm layer of the test tube. A suitable number of sperm 20 may be greater than or equal to 500 or greater than or equal to 1000. A suitable number of sperm 20 may range from about 5,000 to 10,000. However, the suitable number of sperm 20 may be fewer than 500 or greater than 10,000.

Alternatively, male and female sperm can be differentiated by a dual-color fluorescence in situ hybridization technique. Such technique may employ two different colors of LEDs (or from other light sources) to obtain images at high speed without the need to add fluorescence markers that may adversely affect the viability of sperm 20 for fertilization. The sperm of the desired genetic sex can be extracted.

Regardless of whether a sort based on genetic gender is conducted, one may optionally perform some pre-sorting analysis of the donated sperm. For example, a sensor, such as an image sensor or, particularly, a video camera, can be used to collect sperm data in conjunction with computer assisted sperm analysis (CASA) software to evaluate sperm motility, concentration, and/or other sperm factors or characteristics. Such analysis may provide data to help estimate the time period for sorting, as later described. (The CASA software may form part of a software processing system that may utilize an Al system, as later described.)

Such analysis may be employed for a preliminary sort that is not based on genetic sex. For example, a preliminary sort may be employed to discard defective sperm 20. In particular, lack of forward motility may be an indicator of defective sperm. One may appreciate that 30 to 70% of all sperm 20 may exhibit lack of forward motility, depending on the amount of time since they were ejaculated, the health of the mammal donating, the overall fertility fitness of the mammal donating, and the age of the mammal donating.

One will appreciate that if genetic sex is not to be selected for, then a genetic gender presorting process can be omitted. Accordingly, sperm 20 that is not presorted based on genetic sex, or sperm 20 from both layers, can be placed in a single sorting track 18 at an initial placement location 26 such by injection through an entry port 30.

In some embodiments, female sperm 20 may be placed at a first initial placement location 26 such by injection through an entry port 30, and with male sperm 20 may be placed at a second initial placement location 26 that is in a different location in the sorting track 18 such by injection through the same or different entry port 30.

If genetic gender is a selection factor, then sperm 20 from the appropriate layer can be placed at the initial placement location 26 in the sperm sorting track 18, such as with the mini syringe 100 through the injection area 44. If embryos for children of both sexes are desired, then sperm from the different genetic sorting layers can be placed into separate sorting tracks 18. These sorting tracks 18 can be substantially identical, or they can be intentionally different.

For example, a sorting track 18 that is specific for male sperm 20 may be configured to have cubicles 22 positioned more closely together, such as with less distance between the valves 76. Or, a sorting track 18 that is specific for female sperm 20 may be configured to have cubicles 22 positioned more closely together. Moreover, sorting tracks 18 that may be specifically configured for receiving male sperm 20 may have cubicles 22 with other different cubicle characteristics than those used to sort female sperm 20. One will also appreciate that a sperm donation may be divided into separate portions and the separate portions may be sorted on sorting tracks 18 that are intentionally different with respect to their architecture or other cubicle characteristics.

One will also appreciate that a different specific sorting track 18 may be selected for sperm 20 under different specific variables. For example, a scaled down version of a sorting track 18 may be employed for sperm that has been stored, older sperm, or sperm from older donors. After 24 hours, straight line velocity went from 150 to only 55-70 micrometers/sec (and forward motility decreased from 70-80% of motile sperm down to only 30%). Thus, the age of the sperm or the age of the donor may determine the size or type of sorting track 18 that is employed.

Once the sperm 20 have been placed at the initial placement location 26 in the sorting track 18, the sperm 20 can react to various conditions, including the cubicle characteristics, that exist within the sorting track 18 and may become trapped in the cubicles 22. The period of time provided for the sperm 20 to sort may depend on a number of variables. If sperm 20 of only one genetic gender is selected, then the time period may be altered if appropriate. Differences in semen volume may influence the desirable sorting period. Moreover, the swimming speed or forward motility of sperm 20 from every donor may vary. Sperm concentration may also impact sorting time or the quality of a sort. For example, too many sperm per square unit of volume can block the effect of a barrier to sort, and some sperm 20 may ride over top of others, not “feeling” an effect.

One will appreciate that although these criteria can be established prior to placement in the sorting track 18, image sensors such as microvideo cameras can also be employed inside the sorting track 18. Alternatively, or additionally, the sorting track 18 may have a shell material, or windows, through which a video camera can monitor the movement of the sperm 20 for evaluation by the software processing system for computer assisted sperm analysis. Moreover, fluorescence data may be able to be acquired through a variety of materials. Finally, internal features, such as paddle wheels, can be connected to sensors to calculate (such as by measuring the number of times a paddle wheel has spun) may be used to collect sperm data in conjunction with software to evaluate sperm motility and/or concentration for determining an appropriate sorting period.

An average wait time period may be greater than or equal to 10 minutes, 15, minutes or 20 minutes for sperm 20 to sort. An average wait time period may be less than 45 minutes, 40 minutes, or 35 minutes. One will appreciate, however, that an average wait time period may be less than 10 minutes or greater than 45 minutes. Based on camera observations (changes in turbulence of the medium inside of a given cubicle 22, the number of sperm 20 passing through doors to cubicles 22, concentration of sperm in a cubicle) and other sensor data (changes in acidity or electric charge), the average time period for sorting can be adjusted. Moreover, the actual time period employed for a particular sperm-sort event can be determined based on the average wait time as adjusted by these and other factors.

Sensors, such as cameras or lasers, can be employed to count the number of sperm 20 in cach cubicle 22. Lasers, chemicals, or mechanical methods may be employed to limit access to cubicles 22 after enough sperm 20 have entered a given cubicle 22. Thus, in some embodiments, slower sperm 20 could be forced to sort themselves into farther cubicles 22. This limit number of sperm 20 per cubicle 22 can be based on a formula that considers time since start and overall sperm count per unit volume.

After the selected time period, the sperm 20 from some or all the cubicles 22 can be analyzed. For examples, cubicles 22 that are known from past sorts to contain sperm with defective DNA, such as DNA that leads to birth defects or disabling diseases, may be omitted from analysis. The analysis may optionally start with an analysis of sperm data concerning the visibly identifiable characteristics (physical characteristics and capabilities) of the sperm 20. The physical characteristics may include size, shape, and/or other physical characteristics. More particularly, the physical characteristics may include one or more of overall size, overall length, head features including size and/or shape of various sections, and tail length. For example, the head length of sperm may vary about 20 percent, the mid-section which houses the mitochondria may have higher variations, and the flagellum (tail) length may also vary by about 20 percent. These variations may be categorized into different sets.

Sperm capabilities may include swimming style such as speed, direction, circling tendency, and/or grouping tendency. In some embodiments, the sperm capabilities are measured or observed under similar conditions. These conditions may be environmental or ambient conditions. These conditions may be equivalent treatment of sperm 20, such as centrifuged (or not), presence or concentration of extender solution, and recently thawed or thoroughly warm. Sperm capabilities will differ under different conditions. For example, recently thawed sperm 20 move in a sluggish fashion compared to a thoroughly warmed up sperm 20.

Additionally, the electrical activity (electrophysiology) of the sperm 20 themselves may be recorded by automated or manual patch clamping methods to provide an additional characteristic criterion that may be useful for genetic sorting. See https://en.wikipedia.org/wiki/Automated_patch_clamp.

If the sperm 20 in a cubicle 22 is pre-characterized into physical groups and/or capability groups, a test sample including a test number of sperm 20 may be taken from each group within the cubicle 22, such as by a mini extraction syringe 110 through the extraction arca 54 of the extraction portal 50.

In general, the test number of sperm 20 may be greater than or equal to 3 or the test number of sperm 20 may be greater than or equal to 5. In some embodiments, the test number of sperm 20 may be in a range between greater than or equal to 3 and less than or equal to 100. The test number of sperm 20 may be in a range between greater than or equal to 3 and less than or equal to 50. The test number of sperm 20 may be in a range between greater than or equal to 3 and less than or equal to 25. The test number of sperm 20 may be in a range between greater than or equal to 3 and less than or equal to 15. One will appreciate that the test number of sperm 20 may be impacted by the total number of sperm 20 in a group within a cubicle 22 and/or the total number of sperm 20 within a cubicle 22. One will also appreciate that the test number of sperm 20 may be different for different cubicles 22.

In some embodiments, a test number of sperm 20 may be extracted from a cubicle 22 without pre-characterization into groups of physical attributes. In such embodiments, the sperm 20 in this test sample may be characterized for physical attributes (morphology) and/or capabilities (movement signature) after extraction and subsequently sorted into groups of physical attributes. In other words, before DNA analysis of the sperm 20 is initiated, a camera or other sensors can be employed to capture shape, size, swimming style (speed, direction, circling tendency, and/or grouping tendency), and/or other physical characteristics and/or capabilities. This can all be automated and guided by cameras (and/or other sensors) in coordination with a software processing system that employs software running on one or more processors that may serve as an artificial intelligence (AI) system, such as a neural network.

The DNA from the sperm 20 from each test sample is then analyzed for genes using conventional technology developed specifically for analyzing DNA from sperm. Mammalian sperm chromatin is highly condensed. so isolating DNA from such chromatin can be a formidable task. In particular, sperm DNA is highly compacted by the replacement of histones with sperm-specific low molecular weight proteins called protamines. Both the protamines and the disulfide bridges formed within and between protamines inhibit the extraction of high-quality sperm DNA by standard techniques used for somatic cells.

In one embodiment of a sperm DNA analysis method, the lysis buffer may include guanidinium, sodium citrate, sarkosyl, proteinase K, and mercaptoethanol. Proteinase K can also be optionally excluded. CsCl centrifugation of the lysate can be performed or omitted. Instead of centrifugation of the lysate, isopropyl alcohol may be added directly to the lysis buffer to harvest the DNA. Because the modified guanidinium method is a simple one-step procedure that avoids homogenization, organic solvents, centrifugation and produces degradation-free DNA, this method or variations of it could be employed in a home, sperm DNA-analysis kit. See “Modified guanidinium thiocyanate method for human sperm DNA isolation,” Hossain, A. M. et al. Molecular Human Reproduction Vol. 3 (No. 11;953-856 (December 1997). In addition, this guanidine thiocyanate method reported by Hossain can be supplemented with additional modifications resulting in high molecular weight DNA of high quality with an A260/280 ratio ranging between 1.8 and 2.0 and an A260/230 ratio of 2.0 and greater. The DNA is efficiently digested with restriction enzymes and amplified by PCR. See “Methods of sperm DNA extraction for genetic and epigenetic studies,” Griffin J., Methods Mol Biol 2013;927:379-84.

Moreover, sperm cells may be homogenized with 0.2 mm steel beads for 5 minutes at room temperature, using a commercially available method yielded silica-based spin column, in the presence of guanidine thiocyanate lysis buffer supplemented with 50 mM tris (2-carboxycthyl) phosphine (TCEP).

This protocol has many advantages: it may be conducted at room temperature; lengthy proteinase K (ProK) digestions may be eliminated; the reducing agent, TCEP, is odorless and stable at room temperature; nucleic acids are stabilized, allowing storage of homogenate; and it is adaptable for other mammalian species. See “Rapid method for the isolation of mammalian sperm DNA,” Wu, H. et al., Biotechniques (2015 Jun. 1);58(6): 293-300. doi: 10.2144/000114280.eCollection 2015 June. The benefits of these methods may simplify the procedures for circumstances and settings, such may be encountered for home testing kits, where sample processing constraints exist.

The DNA can be analyzed for the test sample as a whole, for a group of sperm 20 within the test sample, or for each individual sperm 20 in the test sample. The later could provide the most information that might ultimately assist the AI system correlate genetic characteristics with specific cubicle characteristics and identifiable sperm characteristics. Using conventional technology, the DNA analysis will kill the sperm 20 in the test samples. Although each test sample can be characterized by all known genes that the sample contains, the test sample may be characterized by the presence (and prevalence) of selected genes. If the sperm 20 in a test sample from a given cubicle 22 are determined to be similar enough genetically, one can make decisions on whether or not to use a sperm 20 from that cubicle 22 to make an embryo (and ultimately a baby).

Genetic similarity may entail that several important genes in a priority list (such as selected by prospective parents) are the same. The number of these matching important genes may be 3 or greater, 5 or greater, 10 or greater, or 25 or greater. One will appreciate that the list may be arranged by order of importance to prospective parents, and that the matching genes can be provided with weighted values to facilitate the evaluation of whether to use sperm 20 from a selected cubicle 22 or selected group from a cubicle 22.

Selected genes may include genes that are defective or code for defects, diseases, disorders, or disease susceptibility. Selected genes may also code for desirable traits. Desirable traits may include traits that are beneficial to health or longevity. Desirable traits may include physical traits, such as height, eye color, hair color, skeletal features, and/or facial features. For example, skeletal features may differentiate between: long torso and shorter lower legs (football defenseman) vs short torso and long lower legs (runway model); square shoulders vs sloped shoulders; big feet vs small feet; high arches vs flat arches; wide feet vs narrow feet; and/or straight profile (athletic) vs curves. These genes can be sorted singly and/or as sets.

Facial features can be generally constructed from DNA information. See “Under the Skin,” Wang, Boen, Daily Collegian, Penn State (https://www.collegian.psu.edu/news/campus/article_ce94ae36-3ca8-11e5-a4ba-University, 832fcedf6fcb.html); “Modeling 3D Facial Shape from DNA,” Claes, Peter et al. PLOS Genet 10(3): e1004224. https://doi.org/10.1371/journal.pgen.1004224; “Recreating the Face of a Perpetrator from a Drop of Blood,” Community Newsletter, (https://verogen.com/wp-content/uploads/2018/08/shriver-pennstate-interview-forensics-1470-2015-001.pdf); “An early modern human from Romania with a recent Neanderthal ancestor” Fu, Qiaomci et al., Nature. 2015, 10.1038/nature14558; “Toward DNA-based facial composites: preliminary results and validation,” Claes, P et al. Forensic Sci Int Genet. 2014, 13:208-216; “DNA as a Sketch Artist: How Forensic Science Benefits from Physical Predictions,” Voss, K., Forensic Magazine, May 2015; “3-D model links facial features and DNA,” Messer, Andrea, Penn State, (https://www.sciencedaily.com/releases/2014/03/140320173321.htm); and “How Accurately Can Scientists Reconstruct a Person's Face from DNA?,” Curtis, Caitlin et al., The Conversation, smithsonianmag.com (https://www.smithsonianmag.com/innovation/how-accurately-can-scientists-reconstruct-persons-face-from-dna-180968951/). By jointly modeling sex, genomic ancestry, and genotype, the independent effects of particular alleles on facial features can be uncovered. Techniques for detecting ancestry informative marker polynucleotides that contain a single nucleotide polymorphism are described in U.S. Pat. No. 6,506,568 and U.S. Pat. Appl. Nos. 20020098484 and 20070037182, which are herein incorporated by reference.

Facial features may also or specifically categorize: straight, convex, or concave nose; thicker or thinner lips; more or less pronounced check bones; whether or not the chin protrudes or is sunken back; size and shape of eyes; spacing between eyes; width of jaw; soft white teeth or hardy yellow teeth; relative shape of the head; relative shape of the face; relative orientations and size ratios of various sets of features; and/or a tendency toward symmetry (inbreeding can tend to stray from symmetry). Relative aspects may include top of the face to bottom of the face. Moreover, sorting criteria might consider eyes to be more widely spaced if correlated with a wider more Neanderthal mouth or eyes more closely spaced eyes if correlated with a smaller, more ET the extraterrestrial mouth. One the other hand, potential parents might also want to avoid an ET mouth altogether because a small mouth may be likely to require teeth being pulled, etc.

Other desirable traits may include dispositions that facilitate aptitudes or abilities, such as endurance, IQ or other intelligence factor, metabolism, and/or male fertility. For example, defects in a gene called DEFB124 can increase male infertility. Metabolic categories may help differentiate between fast burn muscles vs slow burn muscles (sprinter vs marathoner). Customer defined desirable traits might also include the ratio of eye spacing to mouth width, height of head to width of head, ratio of forehead spacing between eyes and mouth, square or pointy chin, large or small eyes, color/texture of hair, color/shape of eyes, size/shape of lips, and/or ability of skin to tan. As noted earlier, these traits may be established by single genes or sets of multiple genes.

In addition, to correlating genetic characteristics to cubicles 22 or groups within cubicles 22, the software processing system (optionally employing an AI system) correlate the genetic characteristics with the identifiable physical characteristics and/or capabilities for the sperm in each sample.

In some embodiments, groups or test samples (and the cubicles 22 where they were obtained) that exhibit genetic defects can be removed from consideration and further analysis. Based on this analysis, one or more cubicles 22 (or one or more groups within one or more cubicles 22) can be selected as a potentially desirable source for sperm 20 to be used for egg fertilization. However, additional optional analysis as later described can be employed before cubicles 22 or groups are selected.

If sperm 20 with desirable genetic qualities are in a cubicle 22 that contains a mix with other sperm 20 that are not close enough genetically, some sperm 20 can be extracted from that cubicle 22 and run through a secondary sorting track 18. For example, 10-20 most important genes (potentially ranked in order to allow for weighted values) can be specified. If a minimum number of these important genes match in each given cubicle 22 yet vary from those that are being paired off in other cubicles 22, then a secondary sort may be not warranted. However, if inadequate number of important genes match, then a secondary sort may be warranted. This minimum number threshold may be dynamic, allowing for a sort to be useful.

The secondary sorting track 18 may have different cubicle characteristics than those of the initial sorting track 18. For example, the secondary sorting track 18 may be smaller, it may have a spiral in the opposite Coriolis direction, or it may utilize different stimuli or characteristics. For a secondary sort, simply the distance and tendency for a sperm to be a left-hand oriented or right-hand oriented with respect to the sorting track 18 may be sufficient for a secondary sort. If a second sorting is conducted, then a second round of test sampling and analysis can be conducted.

A prospective mother's DNA can also be analyzed. One can use DNA from an egg (or from a composite of multiple eggs), which is as close as one can estimate to the egg that may actually be used with a selected sperm. Unfortunately, like with sperm, one cannot know DNA ahead of time, because the egg is a single cell, which would have to be destroyed to analyze the DNA. In another embodiment, one can also take the mother's DNA and run meiosis simulations. In yet another embodiment, one can also use DNA from one or more children of the mother and subtract the DNA from the father to establish the mother's egg contribution, which may be useful for providing statistical weight. Moreover, one can employ multiple ones of these embodiments, providing the results from each embodiment with appropriate statistical weight and combine the results.

A software processing system may be employed to perform any or all of the following described operations. The software processing system may employ software running on one or more processors that may serve as an Al system. The AI system may include one or more of a neural network, a probabilistic technique such as Bayes or Markov algorithm, a kernel method (like support vector machine (SVM), decision trees/random forest, Gaussians, principal component analysis (PCA), or can-cor, etc.), reinforcement learning that can have nothing to do with artificial neural networks, artificial reasoning a.k.a. “good old fashioned Al” (GOFAI), many path-planning and intelligent control-systems methods that correspond to “classical AI” (not the same as GOFAI), alife (swarms, cellular automata . . . ), agents and chaos systems, and/or any algorithm or group of algorithms that optimize a value function (reinforcement learning and linear dynamic programming).

In one embodiment, a spreadsheet program can be created that allows for column sorts for genetic characteristics based on single genes and/or groups of genes, which may be designated by identifiers or headers, such as cubicle 22 or group, hair color, eye color, height, IQ. Examples of suitable spreadsheet programs include, but are not limited to, Excel™, Google Sheets™, Spreadsheet Server™, Smartshect™, Numbers™, and Intellimas™.

Groups of genes may fit statistical data from living people. For example, an IQ score might include an equation that uses 12 or more or 14 or more genes as inputs. Researchers recently pinpointed 52 genes associated with intelligence. Particular IQ genes in these sets might include: SHANK3, which is involved in the formation of the synapses; DCC, which is involved in guiding the growth of axons; and ZRHX3, which regulates the differentiation of neurons from other cell types during development. See “52 Genes Linked to Intelligence,” May 23, 2017, Section D, Page 1 of the New York edition of the New York Times (https://www.nytimes.com/2017/05/22/science/52-genes-human-intelligence.html)

For each test sample, one can use a composite score in which each gene “votes.” The “winner” for the composite may be the gene that tallies the most of votes. For example, if the test sample has three sperm, then the “winner” for the composite would be 2 out of 3 or 3 out of 3. Other examples may be 3 out of 4, 4 out of 5, or 6 out of 7. One will appreciate that any other suitable threshold may be used, and the higher the percentage increases the likelihood that the gene is a characteristic of the group from which the sample was extracted. One will also appreciate that the size of the sample taken for analysis may be limited by the number of sperm 20 in a cubicle 22 or group within a cubicle 22.

The processing software can “push below the line” (eliminate from consideration) any cubicles 22, or groups within cubicles 22, or physical sperm attributes and/or sperm capabilities that may be correlated with genes associated with defects, such as genetic diseases, genetic disorders, single-gene inheritance disorders, multifactorial inheritance disorders, cancers, addictions, and skeletal defects, etc.

Some examples of single-gene disorders include: cystic fibrosis (a mutation in the CFTR gene), alpha-and beta-thalassemias, sickle cell anemia (a mutation in the hemoglobin-Beta gene found on chromosome 11), Marfan syndrome (a mutation in the gene that determines the structure of fibrillin-1), fragile X syndrome (a mutation in the FMRI gene), Huntington's disease (a mutation in the HTT gene), and hemochromatosis (a mutation on the HFE gene).

Some examples of multifactorial inheritance disorders include: groups of genes that influence susceptibility to heart disease, high blood pressure, Alzheimer's disease, arthritis, diabetes, cancer, and obesity. For example, genes associated with breast cancer susceptibility have been found on chromosomes 6, 11, 13, 14, 15, 17, and 22. In another example, some of the 108 genes linked to schizophrenia, or some threshold number of them, may be cause to “red flag” a test sample.

Some examples of chromosomal defects may include: Turner Syndrome, Klinefelter syndrome, and Cri du chat syndrome. Skeletal defects might include Morton's toe. One will appreciate that not all of these listed genetic disorders may qualify for automatic elimination from consideration. However, these diseases and susceptibilities can be weighted, perhaps with the most severe being eliminated. Combinations of multiple potential less severe defects may also be evaluated for removal.

Defective or potentially defective genes, physical sperm attributes and/or sperm capabilities may include attributes of the sperm of other men who had issues with fertility or mutations. In some embodiments, this comparison can be delayed and later compared with candidate sperm for fertilization as a subsequent elimination procedure (or a subsequent positive selection feature in cases where compared sperm exhibited positive factors or did not exhibit fertility issues or mutations. Thus, even though these items may be pushed below the line, some or all of them can still be sorted in the spreadsheet program. For example, it might be useful to know cubicles 22, groups within cubicles 22, physical attributes, or capabilities of defective sperm so they these attributes can be avoided while selecting specific sperm from a desirable cubicle 22 or group. These various factors can be cumulatively stored in a database. Deep learning through an Al system can increasingly sort the variations and identify sperm qualities to be eliminated for consideration and identify sperm qualities to be proactively selected for. Ultimately, the correlated characteristics can be utilized to select a sperm 20 for fertilization of an egg by choosing a cubicle 22 and identifiable sperm characteristics that correlate with desired genetic characteristics.

For each cubicle 22 or group within a cubicle 22, an image (such as a lifelike image or near lifelike cartoon), which is a closest approximation to the final child, can be formulated based on the male genetic input factors. In some embodiments, additional input factors may include female genetic input factors as established by egg DNA or blood DNA from the female donor and her siblings or other relatives. In some other embodiments, the gene analytical data can be combined with photographs of the sperm donor (and/or his relatives) and/or the egg donor (and/or) her relatives to provide very good approximations.

In some embodiments, a variable slider or other means may be employed to provide a progression of forecast images over an age range, such as 1-99, 2-70, 3-55 or other ranges, or forecast images for multiple predetermined ages, such as 2, 5, 10, 15, 20, 30, 50, and 70. One will appreciate the number of predetermined ages may be more than 2 and fewer than or equal to 10 or may be more than 10, and the ages may be different.

Each progression of one or more forecast images may provide a single view or multiple views. These views may be facial images and/or full body images. For example, a four-view embodiment might include: front face, side head, front body, and side body. One will appreciate that these forecast images are only best approximations to provide an idea and that the actual child may have features that will vary. Optionally, clothing style, hair style, and other apparel, such as glasses or hats, can be added. In some embodiments, two or more of these progressions of forecast images can be open and synched, so that a user can scroll the selected progressions at the same time.

In some embodiments, and option to switch, such as with toggles, between one or more of composite DNA, individual DNA (egg DNA or sperm DNA), the manner in which the “egg” DNA is established, and with or without additive components of relative photographic images (as composites of particular relatives or from a single specific relative).

One will appreciate that a variety of selection methods can be utilized based on the genetic data with or without extrapolated and/or weighted data. The software processing system may (or may not) utilize an Al system with deep learning to formulate the forecast images. A user may utilize the software processing system to determine one or more favorite sperm from one or more cubicles 22 or groups within cubicles 22.

If one type of sperm 20 from a test sample from a cubicle 22 or group within a cubicle is slightly better, a controller can choose a sperm 20 from the cubicle 22 for intended fertilization that cross correlates with it, such as looks and swims most like it. As noted earlier, if a favorite sperm type, as determined from a test sample, is in a cubicle 22 that includes a mix of other sperm 20 that are not close enough to the favorite sperm type, some sperm 20 can be extracted from that cubicle 22 and run through a smaller gauntlet sorting track 18 as previously described.

More particularly, the pre-recorded dimensions and movement (parsed by clustering examples such as based on speed, direction, and frequency of oscillation, bouncing off of others, circling and other sperm capabilities as previously discussed) of the sacrificed test sample sperm 20 selected for qualities for fertilization can be matched with remaining sperm 20 from the preferred cubicle 22, sub-cubicle, or group. Surviving sperm 20 taken from the preferred cubicle 22, sub-cubicle, or group may be filmed, and their physical attributes and capabilities can be determined. The surviving sperm 20 that correlates most closely to the most favored sacrificed sperm 20 may be chosen for fertilization. If there is no single best candidate, all individuals may be awarded points based on how closely they correlate to each of the sacrificed individuals from the test sample or on how closely they correlate to them as a group.

Ultimately, a single sperm 20 (or set of sperm 20 depending on the intended fertilization process) that closely matches the favorite sperm type(s) may be selected for fertilization with an egg from the female donor. In some embodiments, intracytoplasmic sperm injection (ICSI) may be employed for direct injection of a single sperm 20 into a single egg to produce an embryo, presumably having the genetic information for a desirable child. This ICSI process may be repeated for a predetermined number of embryos using separate favorite sperm individuals and available eggs to provide a stockpile of embryos if additional children may be desired contemporaneously or over time.

The sorting track 18 may be manufactured and provided as one or more separate items and in many different varieties of shapes and sizes. For example, the outer shell 28 (with inner barriers 30 and valves 76) may be provided as a stand-alone item. The user may provide the various non-structural cubicle characteristics and the injection and extraction devices. In some embodiments, the sorting track 18 may be provided as a sperm-sorting kit with some or all of the cubicle characteristics included as well as the injection and extraction devices. For example, a sorting kit may include a housing that supports an injection area and multiple cubicles, including at least first and second cubicles that have a different combination of cubicle characteristics within the housing, wherein the cubicle characteristics include two or more of: distance from the injection area; size of cubicle opening; cubicle side of sorting track; wavelength in, or near the opening of, the cubicle; light intensity in, or near the opening of, the cubicle; gradient of electromagnetic field in, or near the opening of, the cubicle; chemical treatment in, or near the opening of, the cubicle; and temperature in, or near the opening of, the cubicle. The openings for the cubicles 22 may be close to the order or magnitude of sperm diameter; and the openings may be able to be reliably opened and closed when commanded, based on a count of sperm that have already entered. The kit may also include a sperm collection container and a sperm collection syringe.

Some of the cubicle characteristics, such as some or all of the chemicals, may be supplied in separate containers (e.g. vials or mini vials). Some kits that include a sorting track 18 that uses electrical power for some of its cubicle characteristics may include an electrical connector as previously described. Some versions of the kits may provide better results if they are kept refrigerated. Some versions of the kits may be best utilized by a medical institution or a lab.

However, sorting track kits can be configured for home use. These kits may be more “user friendly” than kits sold to institutions or businesses. Some optional steps or treatments may be omitted. For example, a kit employed by a home user may be sent for certain aspects of processing by a lab or other business. In particular, instead of providing a mini centrifuge or micro-spin apparatus, a lab may handle a gender sort. In one example, a home sorting kit could be configured to run a sperm sort based on an ejaculate with an extender. Cubicle characteristics could still include chemical and electrically based stimuli. The utilized sorting track 18 could be mailed (refrigerated or unrefrigerated, depending on a number of variables) to a lab or medical practitioner's office where further sorting or analyses could be conducted. The patient (or couple) could then visit the practitioner's office (perhaps the office of an OBGYN or the person who would perform the ICSI) to utilize the processing software system with or without the aid and/or advice of a genetic specialist.

In other embodiments, the sperm sorting kits could be configured to provide software (or a link to software or a communication nexus configured for operable connection to processing software) and sperm analyzer that can analyze the DNA from the cubicles 22 and then formulate the forecast images.

One example of a sorting kit may contain necessary items to perform one or more assays per a target number of cubicles 22 groups. A target number of assays may be from about 10 to about 100, about 15 to about 50, or about 20 to about 40. Some of the components of a kit may include: coated slides, Eppendorf agarose tubes, one or more sperm extenders, a denaturant agent, a lysis solution, a restriction enzyme, a float, disposable micropipettes, and instructions for use. The lysis solution may contain guanidinium, sodium citrate, sarkosyl, proteinase K, and mercaptoethanol. Isopropyl alcohol may be added directly to the lysis buffer to harvest the DNA without a centrifugation step. One will appreciate that some or all of the lysis solution components may be supplied separately or that some or all of the components compatible for long term comingling may be provided as separate solutions that may be mixed together by a user within a prescribed period before use.

If three or more sperm 20 are analyzed as a single test sample, then DNA amplification may be omitted. The larger the number of sperm 20 in a test sample may decrease the amount of statistical noise potentially caused by impurities (such as acquired during sperm collection). However, if the DNA of sperm 20 is to be analyzed one at a time, then the DNA may be subjected to PCR or other DNA amplification technique. For such amplification, the test kits may also include template DNA, DNA polymerase, primers, deoxynucleoside triphosphates (dNTPs), cofactor: Mg2+, and buffer. See https://www.thermofisher.com/us/en/home/life-science/cloning/cloning-learning-center/invitrogen-school-of-molecular-biology/pcr-education/pcr-reagents-enzymes/pcr-component- considerations.html. Comparison of the single sperm DNA to the DNA of other single sperm 20 may be used to statistically determine whether the single sperm DNA application was too noisy. If so, a new test sample can be obtained and processed.

In an embodiment of a higher-cost kit that may be configured for repeated use by a customer. Such kit may include a machine and software that automates the entire process (with or without DNA amplification) and generates results to display on a computer. The customer would only donate sperm 20 into an open container, add (provided in a packet or a vial or bottle) an extender at a preset temperature, gently shake the container until the contents are mixed, draw up some of the mixture into a (provided) syringe, then inject into the machine. A more sophisticated machine may even perform the extender addition and shaking steps.

There may be three major components to the software that comes with the kit. The software may be included, or web links to the software may be provided. The first part of the software shows visual examples (still and video) of how to obtain and inject a donation of sperm 20. The second part may have two paths: one to demonstrate to the professional user how to run their own DNA analysis that may useful for the third part of the software, and the other path may describe how to monitor and maintain the optional, automated machine. This automated machine may be modular and may also generate the cubicle specific straws that can use used to freeze semen for later fertilization. The machine may also be connectable to an automated egg fertilization machine. The third part of the software may take the DNA results of all of the sperm 20 and arrange them in a sortable database. The software may act very much like a row and column sortable spreadsheet with macros, and the results may be saved in multiple formats for analysis elsewhere. The spreadsheet may come with default settings, and with icons that may be clicked for customization and sorting suggestions.

As previously described, sperm 20 can be sorted as individual entries, or a composite, where a majority “wins,” to demonstrate a most likely result for a given cubicle 22. These sperm 20 can be virtually combined with candidate egg DNA to produce potential children. Some of these physical traits, and tendencies toward a talent, personality trait, energy level, disease, or defect, will be straightforward to depict and 100% certain, while others will be algorithmically extrapolated from a database and assigned a probability confidence level and range. Given scanned images of each parent as a reference, DNA variation can be used to display relative differences in the appearance of these children. Some of the views to be shown may be face view, side head view, front full body, and side body. Settings can allow for some choices in clothing (e.g. swimwear to winterwear) and hairstyle (e.g. short to long, bangs/no bangs).

A professional customer, such as a lab, may utilize: a bright field or fluorescence microscope, fridge at about 40 degrees F., incubation bath(s) at about 98.6 degrees F. and about 200-212 degrees F., plastic gloves, glass coverslips (24×24 mm), distilled water, ethanol at 70%, 90% and 100%, microwave oven and fume hood. A solution for bright field microscopy visualization may include a Diff-Quick stain (several trademarks) or Wright solution (Merck 1.01383.0500). A solution for fluorescence microscopy visualization may include fluorochromes for DNA staining. These may entail the use of a phosphate buffer solution pH 6.88 (Merck 1.07294.1000) and/or a mounting medium, such as Eukitt® (Panreac 253681). The professional customer would be able to button select on an order screen what these or items that they would prefer in individual portions for each kit or they would prefer to buy in bulk. The home kit may to provide individual portions (or a larger bottle for many runs) to feed into the automated machine. The home customer may want to invest at various levels and may only be interested in certain limited information.

One will appreciate that a Halosperm® kit suitable for performing sperm lysis is commercially available. However, the Halosperm® kit does not appear to provide tools for sperm analysis beyond basic fertility issues.

### EXAMPLES

Example 1. A sorting track for sorting living sperm, comprising: a housing defining at least two cubicles, an initial placement location, and a channel fluidly coupled to the at least two cubicles and the initial placement location; wherein the at least two cubicles each comprise a unique set of cubicle characteristics, an extraction portal, and an entrance fluidly coupled to the channel; and wherein the unique set of cubicle characteristics of the at least two cubicles cach comprise one or more architectural characteristics selected from the group consisting of: a distance from the initial placement location, a cubicle shape, a cubicle volume, a cubicle position within the housing relative to the channel, an entrance size, and an entrance shape.

Example 2. The sorting track of any one of the preceding examples, but particularly Example 1, wherein the unique set of cubicle characteristics of the at least two cubicles cach additionally comprise one or more stimulus characteristics selected from the group consisting of:

a wavelength of light, a light intensity, a radioactive radiation intensity, an electromagnetic field, a chemical treatment, a pressure, a sound, and a temperature.

Example 3. The sorting track of any one of the preceding examples, but particularly Example 2, wherein the one or more stimulus characteristics are present in the at least two cubicles or at or near the entrance of the at least two cubicles.

Example 4. The sorting track of any one of the preceding examples, but particularly Example 2, further comprising a sorting track controller configured to selectively vary the one or more stimulus characteristics of the at least two cubicles.

Example 5. The sorting track of any one of the preceding examples, but particularly Example 1, wherein the housing defines two to one hundred cubicles.

Example 6. The sorting track of any one of the preceding examples, but particularly Example 1, wherein the cubicle shape may be at least partially conical shape or maze-shaped.

Example 7. The sorting track of any one of the preceding examples, but particularly Example 1, wherein the cubicle shape may comprise inner obstacles.

Example 8. The sorting track of any one of the preceding examples, but particularly Example 1, wherein at least one of the at least two cubicles further comprises a camera.

Example 9. The sorting track of any one of the preceding examples, but particularly Example 4, wherein the housing comprises at least one piezoelectrically activated wall for varying a pressure with the channel or within the cubicle as a stimulus characteristic.

Example 10. The sorting track of any one of the preceding examples, but particularly Example 2, the one or more stimulus characteristics is a chemical treatment, and wherein the chemical treatment comprises a sperm attractant or a sperm repellant.

Example 11. The sorting track of any one of the preceding examples, but particularly Example 2, wherein the chemical treatment comprises Lily of the Valley cardiac glycosides, convallotoxin, convallatoxol, convalloside, convallatoxoloside, convallarin, glucoconvalloside, or convallamarin.

Example 12. The sorting track of any one of the preceding examples, but particularly Example 1, wherein the entrance of at least one of the at least two cubicles further comprises an entrance baffle.

Example 13. The sorting track of any one of the preceding examples, but particularly Example 1, wherein the entrance of at least one of the at least two cubicles further comprises a valve.

Example 14. The sorting track of any one of the preceding examples, but particularly Example 1, wherein the housing further defines an access portal located on a second end of the channel opposite a first end, wherein the first end of the channel is fluidly coupled to the initial placement location.

Example 15. The sorting track of any one of the preceding examples, but particularly Example 1, wherein the channel has a generally spiral shape.

Example 16. The sorting track of any one of the preceding examples, but particularly Example 15, wherein the channel comprises a first channel and a second channel; wherein the first channel spirals in a clockwise direction; wherein the second channel spirals in a counterclockwise direction, wherein, a first cubicle of the at least two cubicles is fluidly coupled to the first channel; and wherein a second cubicle of the at least two cubicles is fluidly coupled to the second channel.

Example 17. The sorting track of any one of the preceding examples, but particularly Example 1, wherein the channel comprises at least one section divider.

Example 18. The sorting track of any one of the preceding examples, but particularly Example 1, further comprising a sorting track medium that occupies at least part of the at least two cubicles, the initial placement location, or the channel.

Example 19. The sorting track of any one of the preceding examples, but particularly Example 1, further comprising a guiding agent.

Example 20. The sorting track of any one of the preceding examples, but particularly Example 19, wherein the guiding agent is a nontoxic sperm attractant, progesterone, CatSper, or a calcium solution.

### CONCLUSION

The terms and descriptions used above are set forth by way of illustration and example only and are not meant as limitations. Those skilled in the art will recognize that many variations, enhancements and modifications of the concepts described herein are possible without departing from the underlying principles of the invention. For example, skilled persons will appreciate that the subject matter of any sentence or paragraph can be combined with subject matter of some or all of the other sentences or paragraphs, except where such combinations are mutually exclusive. The scope of the invention should therefore be determined only by the following claims, claims presented in a continuation patent application, and equivalents to the foregoing claims.

1. A sorting track for sorting living sperm, comprising:
a housing defining at least two cubicles, an initial placement location, and a channel fluidly coupled to the at least two cubicles and the initial placement location;
wherein the at least two cubicles each comprise a unique set of cubicle characteristics, an extraction portal, and an entrance fluidly coupled to the channel; and
wherein the unique set of cubicle characteristics of the at least two cubicles each comprise one or more architectural characteristics selected from the group consisting of: a distance from the initial placement location, a cubicle shape, a cubicle volume, a cubicle position within the housing relative to the channel, an entrance size, and an entrance shape.

2. The sorting track of claim 1, wherein the unique set of cubicle characteristics of the at least two cubicles each additionally comprise one or more stimulus characteristics selected from the group consisting of: a wavelength of light, a light intensity, a radioactive radiation intensity, an electromagnetic field, a chemical treatment, a pressure, a sound, and a temperature.

3. The sorting track of claim 2, wherein the one or more stimulus characteristics are present in the at least two cubicles or at or near the entrance of the at least two cubicles.

4. The sorting track of claim 2, further comprising a sorting track controller configured to selectively vary the one or more stimulus characteristics of the at least two cubicles.

5. The sorting track of claim 1, wherein the housing defines two to one hundred cubicles.

6. The sorting track of claim 1, wherein the cubicle shape may be at least partially conical shape or maze-shaped.

7. The sorting track of claim 1, wherein the cubicle shape may comprise inner obstacles.

8. The sorting track of claim 1, wherein at least one of the at least two cubicles further comprises a camera.

9. The sorting track of claim 4, wherein the housing comprises at least one piezoelectrically activated wall for varying a pressure with the channel or within the cubicle as a stimulus characteristic.

10. The sorting track of claim 2, the one or more stimulus characteristics is a chemical treatment, and wherein the chemical treatment comprises a sperm attractant or a sperm repellant.

11. The sorting track of claim 2, wherein the chemical treatment comprises Lily of the Valley cardiac glycosides, convallotoxin, convallatoxol, convalloside, convallatoxoloside, convallarin, glucoconvalloside, or convallamarin.

12. The sorting track of claim 1, wherein the entrance of at least one of the at least two cubicles further comprises an entrance baffle.

13. The sorting track of claim 1, wherein the entrance of at least one of the at least two cubicles further comprises a valve.

14. The sorting track of claim 1, wherein the housing further defines an access portal located on a second end of the channel opposite a first end, wherein the first end of the channel is fluidly coupled to the initial placement location.

15. The sorting track of claim 1, wherein the channel has a generally spiral shape.

16. The sorting track of claim 15, wherein the channel comprises a first channel and a second channel;
wherein the first channel spirals in a clockwise direction;
wherein the second channel spirals in a counterclockwise direction,
wherein, a first cubicle of the at least two cubicles is fluidly coupled to the first channel; and
wherein a second cubicle of the at least two cubicles is fluidly coupled to the second channel.

17. The sorting track of claim 1, wherein the channel comprises at least one section divider.

18. The sorting track of claim 1, further comprising a sorting track medium that occupies at least part of the at least two cubicles, the initial placement location, or the channel.

19. The sorting track of claim 1, further comprising a guiding agent.

20. The sorting track of claim 19, wherein the guiding agent is a nontoxic sperm attractant, progesterone, CatSper, or a calcium solution.
